(19) United States
(12) Patent Application Publication
(10) Pub. No.: US 2023/0333108 A1
(43) Pub. Date: Oct. 19, 2023

(54) APTAMER-BASED POINT-OF-CARE ASSAY DEVICES AND METHODS

(71) Applicant: The Regents of the University of California, Oakland, CA (US)

(72) Inventors: Drew A. Hall, La Jolla, CA (US); Eliah Aronoff-Spencer, San Diego, CA (US); Naveen Singh, State College, PA (US); Partha Ray, San Diego, CA (US)

(21) Appl. No.: 18/005,178

(22) PCT Filed: Jul. 19, 2021
(86) PCT No.: PCT/US2021/042258

§ 371 Date: Jan. 11, 2023

Related U.S. Application Data
(60) Provisional application No. 63/053,532, filed on Jul. 17, 2020.

(51) Int. Cl. G01N 33/569; G01N 33/543

(52) U.S. Cl. CPC G01N 33/56983; G01N 33/54326; G01N 33/54366; G01N 2333/165; G01N 2469/10

(57) ABSTRACT

Disclosed are systems, devices and methods for a quantitative aptamer-based viral assay. In some aspects, an aptamer-based viral assay device includes a substrate and a biochemical complex conjugated to the substrate, which comprises an aptamer that is initially bound to an enzyme-tagged complementary strand of nucleotides, the aptamer corresponding to an antigen of a virus (e.g., SARS-CoV-2) that has a higher binding affinity to the aptamer than the complementary strand of nucleotides, wherein, when the device is exposed to a solution containing the virus, the enzyme-tagged strand is released from the aptamer as the aptamer binds the antigen of the virus, such that the released enzyme is capable of converting a substance to an analyte that is measurable by a remote analyte meter to correlate with a parameter of the virus in the solution.

### CROSS-REFERENCE TO RELATED APPLICATIONS

This patent document claims priorities to and benefits of U.S. Provisional Pat. Application No. 63/053,532, titled “APTAMER-BASED POINT-OF-CARE TEST FOR COVID-19 USING A GLUCOMETER” and filed on Jul. 17, 2020. The entire content of the aforementioned patent applications is incorporated by reference as part of the disclosure of this patent document.

### TECHNICAL FIELD

This patent document relates to biosensor technologies.

### BACKGROUND

Biosensors can provide real-time detection of physiological substances and processes in living things. A biosensor is an analytical tool that can detect a chemical, substance, or organism using a biologically sensitive component coupled with a transducing element to convert a detection event into a signal for processing and/or display. Biosensors can use biological materials as the biologically sensitive component, e.g., such as biomolecules including enzymes, antibodies, nucleic acids, etc., as well as living cells. For example, molecular biosensors can be configured to use specific chemical properties or molecular recognition mechanisms to identify target agents.

### SUMMARY

Disclosed are systems, devices and methods for a quantitative aptamer-based viral assay for virus identification in a patient sample, which can be implemented at scale. In some embodiments, the disclosed aptamer-based viral assay platform can be configured as a point-of-care (POC) SARS-CoV-2 antigen rapid test, implementable on a low cost analyte meter, including a common glucometer.

In some aspects, an aptamer-based viral assay device includes a substrate including a surface; and a biochemical complex conjugated to the surface of the substrate, the biochemical complex comprising an aptamer that is initially bound to a complementary strand of nucleotides that binds an enzyme at a region of the complementary strand of nucleotides, the aptamer corresponding to an antigen of a virus that has a higher binding affinity to the aptamer than the complementary strand of nucleotides, wherein, when the device is exposed to a solution containing the virus, the biochemical complex is configured to release the complementary strand of nucleotides that binds the enzyme from the aptamer to the solution and bind the antigen of the virus to the aptamer to form a modified biochemical complex conjugated to the surface of the substrate, and wherein the enzyme that is released to the solution is capable of converting a substance to an analyte that is measurable by a remote analyte meter to correlate with a parameter of the virus in the solution. The aptamer-based viral assay device can be implemented as a POC, home-testing, and/or laboratory test for the target virus.

In some aspects, a POC aptamer-based COVID-19 assay device includes a magnetic bead; and a biochemical complex comprising one or both of a nucleocapsid protein (N protein) specific aptamer and a spike surface glycoprotein (S protein) specific aptamer conjugated to the magnetic bead, wherein the one or both of the N protein specific aptamer and the S protein specific aptamer is pre-hybridized with a complementary DNA strand attached to invertase; wherein, upon mixing the device with a sample containing SARS-CoV-2 virus in a container, the complementary DNA strand attached to the invertase is released from the biochemical complex and the one or both of the N protein specific aptamer and the S protein specific aptamer is bound to one or both of N protein antigen and S protein antigen of the SARS-CoV-2 virus, respectively, to form a modified biochemical complex conjugated to the magnetic bead, wherein, upon applying a magnet field at the container, the magnetic bead conjugated with the modified biochemical complex is separated from the invertase attached to the complementary DNA strand, and wherein the separated invertase is able to react with sucrose to generate glucose that is able to be directly measured using a glucometer, wherein the measured glucose is correlatable with a parameter of the SARS-CoV-2 virus in the sample.

In some aspects, a POC aptamer-based COVID-19 assay method includes forming an assay device by conjugating one or both of a nucleocapsid protein (N protein) specific aptamer and a spike surface glycoprotein (S protein) specific aptamer to a magnetic bead and pre-hybridizing the one or both of the N protein specific aptamer and the S protein specific aptamer with a complementary DNA strand attached to invertase; mixing the assay device with a biological sample collected from a patient to test for SARS-CoV-2 virus, wherein the mixing the assay device with the biological sample facilitates binding of the SARS-CoV-2 virus to the assay device by the one or both of the N protein specific aptamer and the S protein specific aptamer binding to one or both of N protein antigen and S protein antigen of the SARS-CoV-2 virus, respectively, and wherein, upon binding of the assay device with the SARS-CoV-2 virus, the complementary DNA strand attached to the invertase is released; applying a magnetic field to the mixture to separate the magnetic bead conjugated with the modified biochemical complex from the complementary DNA strand attached to the invertase; reacting the invertase with sucrose to generate glucose that can be directly read using a glucometer; and correlating a glucose concentration with a parameter of the SARS-CoV-2 virus from the biological sample.

The subject matter described in this patent document can be implemented in specific ways that provide one or more of the following features.

### BRIEF DESCRIPTION OF THE DRAWINGS

FIGS. 1A and 1B show diagrams illustrating example embodiments of a method for a point-of-care aptamer-based viral assay in accordance with the present technology.

FIG. 1C shows a data plot of a calibration graph for an example implementation of a quantitative, glucometer-based point-of-care test for SARS-CoV-2 virus analytes, in accordance with the present technology.

FIG. 1D shows a diagram illustrating an example embodiment of an antisense complementary strand including multiple enzymes per antisense strand.

FIGS. 2A-2E show an illustrative diagram and example data of an example assay validation method to test the release of an oligonucleotide strand upon a target antigen binding to an example aptamer sensor, in accordance with the present technology.

FIGS. 3A and 3B show an illustrative diagram depicting an example conjugation technique and example data plots from electrophoretic mobility shift assay.

FIGS. 4A-4D show data plots depicting example results of an example implementation for detecting N and S protein using an example SARS-CoV-2 assay.

FIGS. 5A and 5B shows data plots depicting example results of an example implementation for detecting N and S protein using a glucometer.

FIGS. 6A and 6B show data plots depicting example results from example implementations for detection of N and S protein in saliva using a glucometer.

FIGS. 7A and 7B show data plots depicting example data from an cross-reactivity, target antigen specificity assay.

FIGS. 8A and 8B show an illustration and data plots depicting example data from example implementations for detection of protein N and S using authentic SARS-CoV-2 virus.

FIGS. 9A and 9B show example data plots depicting COVID-19 clinical saliva samples.

FIGS. 10A and 10B show data plots depicting example results from clinical performance of the example assay using saliva samples from COVID-19 patients and healthy volunteers.

FIG. 11 provides a summary of reported point-of-care diagnostic tests.

FIG. 12 shows a diagram illustrating an example embodiment of a semi-automated test container couplable to an example embodiment of a custom analyte meter, in accordance with the present technology.

FIG. 13 shows a diagram illustrating an example embodiment of an aptamer-based viral assay method using the example semi-automated test container and example custom analyte meter shown in FIG. 12.

FIG. 14 shows a diagram illustrating another example embodiment of a semi-automated test container couplable to another example embodiment of a custom analyte meter, in accordance with the present technology.

FIG. 15 shows a diagram illustrating an example embodiment of an aptamer-based viral assay method using an example embodiment of semi-automated test container.

FIGS. 16A and 16B include data plots listing example antisense (oligonucleotide) sequences configured to bind to the anti-N protein aptamer and to the anti-S protein aptamer, respectively.

FIG. 17A shows an image depicting an example result of a colorimetric control process for indicating the successful addition of saliva to the assay receptacle.

FIG. 17B shows an image depicting an example result of a colorimetric control process for indicating the conversion of the sucrose to glucose and fructose in the presence of the invertase enzyme.

FIG. 18 shows a table depicting a comparison between a low-temperature bacterial invertase enzyme and a high-temperature yeast invertase enzyme.

### DETAILED DESCRIPTION

Lateral flow immunoassay tests (LFIT) are devices intended to detect the presence of a target substance in a liquid sample without the need for traditional, expensive analysis equipment. These test devices are widely used in medical diagnostics for home testing, point-of-care (POC) testing, or laboratory use. An example of LFIT device is a home pregnancy test, which detects a certain hormone associated with a pregnant female from her urine sample. LFIT devices typically operate as an enzyme-linked immunosorbent assay (ELISA), where the LFIT device allows a liquid sample to deposit on a surface modified with corresponding molecules that react in the presence of the target analyte in the sample, in which a reaction is manifested by a visual change indicating a result.

While lateral flow immunoassay tests are attractive for point-of-care rapid antigen testing, they currently lack sufficient sensitivity for certain analytes. In particular, existing POC diagnostic technology has shown poor sensitivity for detection of the Severe Acute Respiratory Syndrome Coronavirus 2 (SARS-CoV-2) virus (also referred to as COVID-19 or COVID-19 virus), and thus remain qualitative and unreliable.

Disclosed are systems, devices and methods for quantitative testing for virus identification in a patient sample, which can be implemented at scale. In some aspects, the disclosed approach provides an inexpensive (e.g., < $1/test), quantitative POC test for COVID-19 diagnosis, which can utilize an existing class of sensor devices, e.g., already ubiquitous in the market today, without the need to develop custom hardware.

For example, there are currently 34 million Americans that rely on a glucometer to manage their blood sugar levels, making the glucometer the most prevalent piece of diagnostic equipment in the U.S. and likely globally. Using a common glucometer, the disclosed technology is able to map SARS-CoV-2 antigen(s) to glucose in a manner to be read by a simple, off-the-shelf glucose meter. The disclosed technique is able to translate COVID-19 viral antigen(s) binding into glucose production, e.g., provided via a novel, engineered, and validated aptamer-based competitive assay.

Example embodiments and example implementations of the disclosed technology are discussed below.

### Example Embodiments

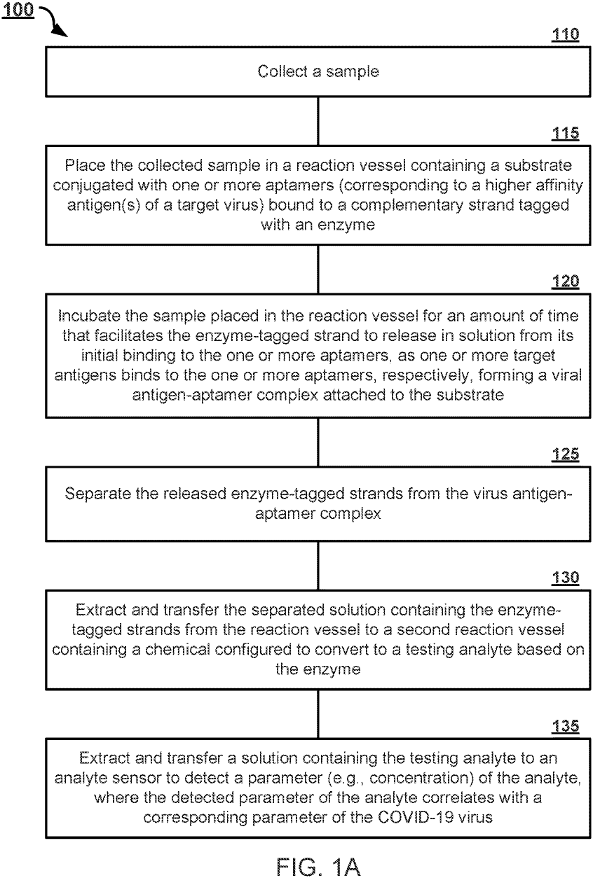

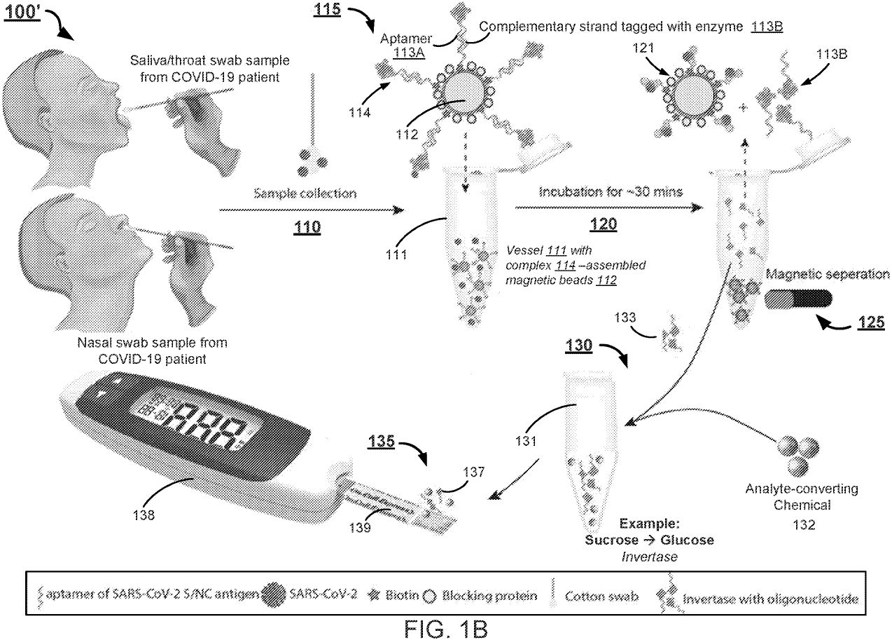

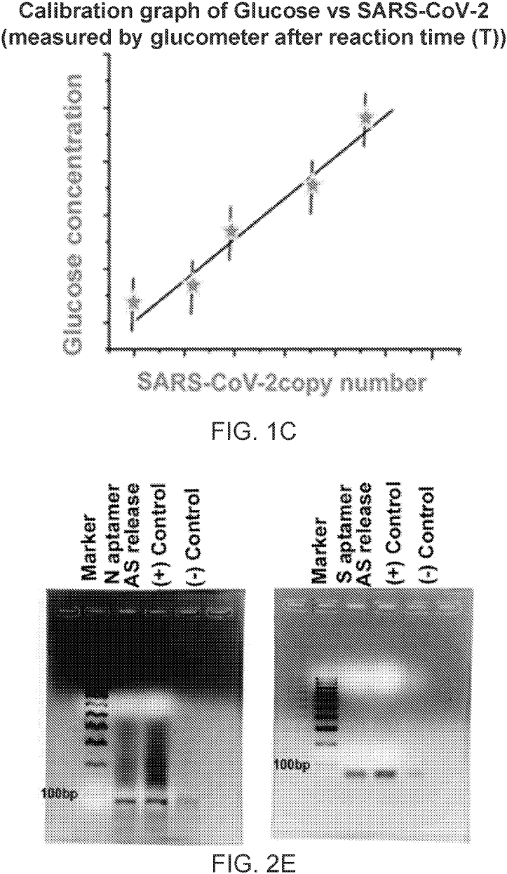

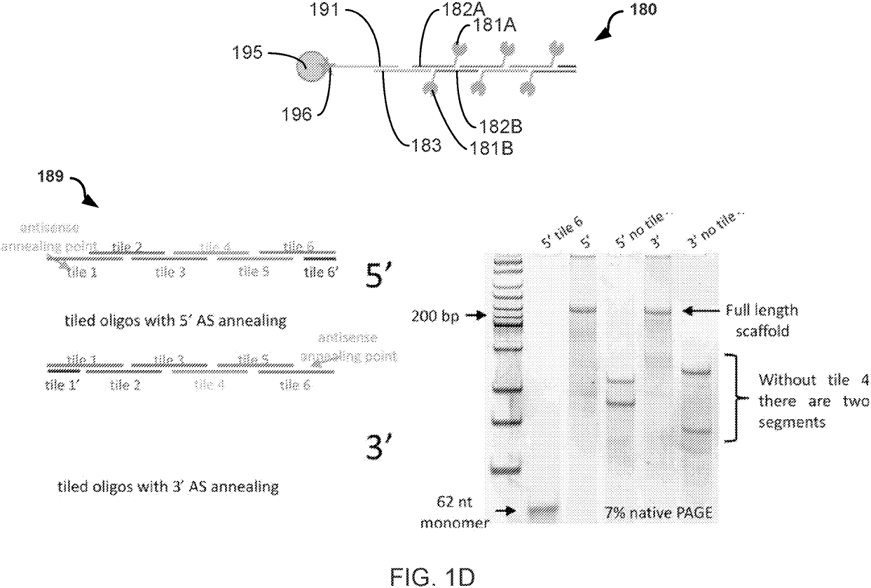

FIGS. 1A and 1B show diagrams illustrating example embodiments of a method 100 for a point-of-care aptamer-based viral assay in accordance with the present technology.

As shown in the block diagram of FIG. 1A, the method 100 includes a process 110 to collect a sample from a patient. In some implementations of the process 110, for example, the sample can include a saliva sample, throat swab sample, or nasal swab sample; notably, for example, collecting patient saliva is likely to be more user friendly for population screening than blood or nasal swabs. The method 100 includes a process 115 to place the collected sample in a reaction vessel containing an aptamer-based viral assay device, which can include a substrate having a surface conjugated with one or more aptamers, which correspond to a higher affinity antigen(s) of the target virus (e.g., SARS-CoV-2 virus), and which is configured to initially be bound to a complementary strand tagged with an enzyme. In some embodiments, the one or more aptamers include a nucleic acid aptamer (e.g., DNA aptamer, RNA aptamer, or XNA aptamer) comprising a strand or strands of an oligonucleotides; whereas in some embodiments, the one or more aptamers include a protein aptamer comprising amino acid residues, which can be attached to a protein scaffold. In some embodiments, for example, the complementary strand can include a small strand nucleic acid sequence, e.g., such as an oligonucleotide. The complementary strand is sometimes referred to herein as the antisense strand (and in the examples of an oligonucleotide strand, an antisense oligo, or antisense oligonucleotide strand, or the like). The surface conjugated with the one or more aptamers initially bound to the enzyme-tagged complementary strand may be referred to as the biochemical complex. In some embodiments of the aptamer-based viral assay, such as for home testing and/or point-of-care testing, for example, the substrate can include a magnetic particle, a polystyrene particle, a gold particle or other solution-suspendable particle that can facilitate conjugation of the biochemical complex, e.g., in which the particle can be a microparticle or a nanoparticle. Whereas, in some embodiments of the aptamer-based viral assay, such as for laboratory testing environments, for example, the substrate can include a well plate, petri dish, or other container structure. The aptamer-based viral assay device can be implemented as a POC, home-testing, and/or laboratory test for the target virus.

In some embodiments, the one or more aptamers that are initially bound to the enzyme-tagged complementary strands include an anti-S protein aptamer and/or an anti-N protein aptamer, which correspond to the SARS-CoV-2 Spike (S) protein and the SARS-CoV-2 Nucleocapsid (N) protein, respectively. In some embodiments (like that shown later in FIG. 1B), the example anti-S (and/or anti-N) protein aptamer(s) is conjugated to the enzyme invertase through a small oligonucleotide that is complementary to a portion of the aptamer sequence. In example embodiments of the anti-S protein aptamer and anti-N protein aptamer, these aptamers include a strand of oligonucleotides that have a high affinity to binding to the S protein and the N protein of SARS-CoV-2. Examples of the anti-S protein aptamer and anti-N protein aptamer are discussed in detail below.

The method 100 includes a process 120 to incubate the sample placed in the reaction vessel for an amount of time (e.g., between 15 to 30 minutes, or in some implementations ~30 minutes), which facilitates the enzyme-tagged complementary strand to release in solution from its initial binding to the one or more aptamers, as the target antigen(s) (e.g., one or more proteins of a target virus, which corresponds to the one or more aptamers) binds to the one or more aptamers of the conjugated biochemical complex. In this manner, the biochemical complex is modified to form a modified biochemical complex that includes the one or more aptamers conjugated to the surface of the substrate and now bound (e.g., hybridized) to target antigen(s). In some implementations of the process 120, for example, the release of the enzyme tagged strand is directly proportional to the binding of the viral antigen.

The method 100 includes a process 125 to separate the viral antigen-aptamer complex bound to the substrate from the enzyme-tagged strands in the solution. In some embodiments, for example, the process 125 includes applying a magnetic field to cause separation of the viral antigen-aptamer complex-bound magnetic beads to pull to a desired region of the reaction vessel away from the enzyme-tagged strands in the solution (e.g., allowing the released enzyme-tagged strand to suspend in the sample solution). In some embodiments, for example, the process 125 includes filtering the solution, e.g., where the enzyme-tagged strands in the solution can pass through pores of a filter and the substrate (e.g., polystyrene particles of a size 1 µm or greater, e.g., 4 µm or 10 µm) that have the modified biochemical complex attached cannot filter through.

The method 100 includes a process 130 to extract and transfer the separated solution containing the enzyme-tagged strands from the reaction vessel to a second reaction vessel containing a chemical configured to convert to a ubiquitous analyte based on the enzyme. In example implementations of the process 130, the second reaction vessel contains sucrose, which converts to glucose based on the enzyme invertase. The method 100 includes a process 135 to extract and transfer a solution containing the testing analyte (from the second reaction vessel) to an analyte sensor for detect a parameter (e.g., concentration) of the analyte, where the detected parameter of the analyte correlates with a corresponding parameter of the target virus.

In some embodiments, the method 100 includes a process (not shown in FIG. 1A) to operate an analyte sensor device to detect the parameter of the analyte. In some implementations, the analyte sensor device includes a glucometer, which detects a concentration of glucose that corresponds to a copy number of the target virus in the collected sample.

As shown in the illustrative diagram of FIG. 1B, an example embodiment of the assay for SARS CoV-2 virus detection works as follows, labeled as method 100′. As shown in the diagram of FIG. 1B, a sample (e.g., saliva or nasopharyngeal swab) is collected from a patient in the process 110. At the process 115, the sample is placed in the example reaction vessel 111, e.g., a vial, that contains surface-modified magnetic beads 112 that are conjugated with anti-S (or anti-N) protein aptamers 113A, e.g., conjugated to the enzyme invertase through a small oligonucleotide 113B (e.g., ~20 base-pair oligonucleotide) that is complementary to a portion of the aptamer sequence. This short complementary oligo with the tagged enzyme invertase 113B is readily displaced by the higher affinity antigen (e.g., S or N protein) of the target analyte, i.e., COVID-19 virus, thus creating an antigen sensitive switch for signal production. The aptamer-oligo-enzyme complex 114 is pre-assembled on the magnetic beads 112 inside the reaction vessel 111.

In some embodiments, the aptamer-oligo-enzyme complex 114 includes biotin at a terminus of the aptamer 113A such that it is opposite the enzyme when the complementary oligonucleotide strand tagged with the enzyme 113B is bound to the aptamer 113A. In such embodiments, the biotin-terminated aptamer-oligo-enzyme complex 114 is immobilized over streptavidin-coated magnetic beads 112. In some embodiments, the streptavidin-coated magnetic beads 112 can also be modified with blocking protein, e.g., to prevent binding of other undesired binding to the magnetic beads 112.

For example, for the implementation of this example embodiment of the process 115, the method 100 can include a process 116 prior to implementation of the process 115, where the one or more COVID-19 specific aptamers (e.g., anti-S protein aptamer and/or anti-N protein aptamer specific to the S and N proteins of COVID-19 virus, respectively) is conjugated to the magnetic bead 112 and pre-hybridized with a complementary DNA (e.g., oligonucleotide) strand attached to invertase, i.e., the example complementary strand tagged with invertase 113B.

In implementations of the process 120, for example, after incubating the sample (e.g., about 30 minutes) in the reaction vessel 111, an amount of enzyme-tagged oligonucleotide strand 113B initially bound to the one or more protein aptamers 113A is released in solution due to the binding of the one or more protein aptamers 113A to the target antigen protein(s) of COVID-19, in which the example invertase-tagged strands are released in an amount or manner directly proportional to the viral antigen.

In some embodiments of the process 125, for example, a magnet can be placed next to the reaction vessel 111 (e.g., vial) to pull down the COVID-19 antigen-bound aptamer complex-attached magnetic beads 121 to a bottom region of the vial, as well as any unbound aptamer-oligo-invertase complex 114 attached magnetic beads 112. The upper portion of the vial will thereby have a solution 133 containing the released enzyme-tagged strand 113B (e.g., invertase-attached oligonucleotides), which can subsequently be transferred to a second reaction vessel 131 containing an analyte-converting chemical 132 (e.g., sucrose) at the process 130.

In some embodiments of the process 130 where the enzyme-tagged strands 113B include the enzyme invertase, for example, the analyte-converting chemical 132 is sucrose, and the extracted solution 133 containing the released invertase enzyme-tagged oligonucleotide strands is transferred to the second reaction vessel 131 (e.g., vial) containing the sucrose. After an incubation time (e.g., ~10 minutes), in which the invertase enzymatically converts the sucrose 132 to glucose (i.e., glucose being an analyte able to be directly readout using an existing glucometer), the a sample 137 from the reacted solution in the second reaction vessel 11 can be transferred to a glucose test strip 139 to perform a test at the process 135. At the process 135, for example, the glucose test strip 139, which contains the sample 137 having the converted analyte glucose (e.g., converted from sucrose via the enzyme invertase), can then be inserted into a glucometer 138 (thus providing the necessary amplification). The glucose concentration detected by the glucometer 138 from the sample 137 is correlated with SARS-CoV-2 copy number.

FIG. 1C shows a data plot of a calibration graph for an example implementation of an example quantitative, glucometer-based point-of-care test for SARS-CoV-2 virus analytes. The example data displayed in the calibration graph is from an example implementation of the method 100, measured by an example glucometer after a reaction time (T), and showing the glucose concentration versus COVID-19 copy number.

A major hurdle in repurposing a glucometer for direct detection of SARS-CoV-2 is that the target biomarkers (e.g., N protein and S protein) are present at low concentrations in biological samples. For example, the average COVID-19 viral load in nasal/throat, sputum, and saliva samples is 3×10⁶, 7.50×10⁵, and 3.5×10⁷ copies/mL, respectively, necessitating signal amplification to generate product (e.g., glucose) in quantities similar to physiological levels in human blood (e.g., 10-600 mg/dL or 0.6-33 mM).

Example implementations of the example platform shown in FIGS. 1A and 1B demonstrated that this approach is capable of outperforming an ELISA technique with N and S protein limits of detection (LOD) of 2.34 pM and 2.64 pM for the N and S protein, respectively, in under 45 minutes using the simple workflow described above. The example studies described herein demonstrated that the disclosed approach is imminently feasible and ready for rapid, mass-scale-up.

Using the disclosed COVID-19 analyte detection technique, for example, a user (e.g., physician, nurse, other healthcare worker, or any individual) can perform a simple sample collection and preparation and execute a COVID-19 virus detection test using a common glucometer, thereby allowing rapid, quantitative detection of COVID-19 in a POC setting.

FIG. 1D shows a diagram illustrating an example embodiment of an antisense complementary strand 180 including multiple enzymes 181 per antisense strand. For example, this embodiment of the antisense strand can further the effect of amplifying the detection signal and allow the assay time to be reduced. As shown in FIG. 1D, multiple enzymes 181A, 181B, etc. (e.g., multiple invertase molecules) are conjugated to the single antisense strand 180 using a branched DNA matrix. In this example, six nucleic acid tiles 182A, 182B, etc., are bound to a base antisense strand 183 that is complementary to the aptamer 191 conjugated to the substrate 195 via conjugation (chemical(s)) 196, where each tile includes one enzyme. This approach, for example, has the benefits of increasing the number of invertase released per binding event, thus cutting the time needed to produce glucose linearly by that same amount. It also has the benefit that the number of invertase per antisense strand is well controlled. Furthermore, this example multiple enzyme-tagged antisense construct can reduce the assay-assay variability. The amount of enzymes that can be conjugated to the antisense are limited by the practical limitations, e.g., from factors like the size and/or mass of the enzyme, the antisense strand or individual tiles, solution kinetics, etc. Also shown in FIG. 1D, a schematic and example data 189 illustrate a “tile-able” strategy where each tile has an invertase conjugated to it, with the example gel data verifying that the number of tiles is controllable via a self-assembly strategy.

### Example Implementations

### SARS-CoV-2 Assay Using a Glucometer

As shown by the example data described herein, the disclosed antigen-switch detection technology allows for detection of SARS-CoV-2 virus using a common glucometer readout. To test the disclosed technique in example implementations, the assay was tested for specifically targeting higher sensitivity and reduced assay time. The cross-reactivity of the assay was assessed against other SARS-like viruses as well as common strains of influenza. A validation study using inactivated virus and bio-banked SARS-CoV-2 saliva (N=5) was also performed. The example results were compared against ELISA. These example implementations allowed for refinement and optimization of the example glucometer-based POC assay for SARS-CoV-2 virus, cross-reactivity assessment against other closely related SARS virus proteins, and clinical testing of the assay using inactivated clinical specimens and bio-banked SARS-CoV-2 saliva.

The example embodiments of the disclosed system, like that demonstrated in FIGS. 1A and 1B, are envisioned to be used to detect infection in COVID-19 patients early in the disease and in individuals with little or no symptoms. Example implementations of the disclosed technique using the engineered anti-S and anti-N protein aptamers optimized for the binding properties of the SARS-CoV-2 anti-S and anti-N protein aptamer(s) to antigen in terms of the limit of detection (LOD) and diminish non-specific binding. These assays were tested with laboratory-validated clinical specimens from infected individuals at different stages of illness, e.g., to show that technology transfers and scales-up for potential global, wide-scale use.

Diagnostic testing to identify people infected with severe acute respiratory syndrome-related coronavirus-2 (SARS-CoV-2) infection is central to control of the global pandemic of COVID-19 that began in late 2019. In the United States, testing is hampered by limited capacity cost and logistics of deployment often leading to prioritized testing for specific high-risk groups. Real-time reverse transcriptase polymerase chain reaction (RT PCR)-based assays performed in a laboratory on respiratory specimens remains the reference standard for COVID-19 diagnostics. Serologic immunoassays are rapidly emerging but are associated with several limitations including sensitivity to detect early infections and reported high false positive rates for some tests. Hence there is urgent need for an accurate and efficient quantitative diagnostic test for detection of SARS-CoV-2 virus in infected individuals. Ideally this test would have the following characteristics: easy to use by an inexperienced tester (e.g., at-home or in the community), be able to reliably detect early (asymptomatic or pauci-symptomatic) infection with a low false positive rate and be sufficiently inexpensive to deploy repeatedly across global populations.

The example glucometer-based aptamer-sensor is uniquely suited to fill a critical global diagnostic need as reagents can be made quickly and scaled cheaply, and glucometers are ubiquitous and available at low cost around the world. The disclosed approach offers following specific advantages and benefits:
Aptamers against SARS-CoV-2 virus will likely be a cheaper alternative to monoclonal antibody-based methods for mass production of POC tests. Additionally, aptamers are stable for extended periods at room temperature, and unlike antibodies, do not require special low temperature storage conditions.
The entire enzyme-antisense strand-aptamer-magnetic bead complex can be lyophilized and stored dry.
A low-cost measurement device (e.g., a glucometer) provides a transformative, scalable solution to perform field- or home-based electrochemical detection of SARS-CoV-2 virus.
The disclosed assay is simple enough to be used by minimally trained users.
The test requires no external resources.
This supports the ability to acquire, process, display, and transmit data to support contact tracing and telemedicine.
Aptamers can also be quickly selected against other viral antigens, and this assay platform can be adopted to detect nearly any other infectious disease while retaining the same readout.

Severe Acute Respiratory Syndrome Coronavirus 2 (SARS-CoV-2) virus, the causative viral agent for the COVID-19 (Coronavirus Disease 19) infection believed to have originated in the Hubei province of Wuhan city in China in late December 2019, has rapidly spread across the whole world. True statistics of this pandemic are difficult to estimate due to significantly limited testing capability; however, the number of worldwide confirmed cases as of July 2020 exceeds 12.8 million and there have been ~0.5 million confirmed deaths. At that time, the United States had the greatest number of cases (over 3.3 million as of Jul. 11, 2020) - ~ 25% of global incidence to date and more than 41 million viral tests have been performed. The SARS-CoV-2 virus is very contagious with a high human-to-human transmission rate and, in the absence of a vaccine or highly effective therapeutic, there is little communities can do to slow down or stop the transmission, such as social distancing and contact tracing, which remain underutilized.

The U.S. is significantly behind in the screening effort due to the lack of cheap, rapid, and accurate diagnostic tests to directly detect the virus. Reverse Transcriptase-Polymerase Chain Reaction (RT-PCR)-based assays are currently the gold-standard for detecting the RNA virus; however, these assays are slow and generally not suitable for large-scale population screening as they require expensive instrumentation and trained laboratory personnel. Recent stories of rationing have been reported further hindering testing. Continued efforts are underway to scale up RT-PCR assays, but throughput will be limited for the foreseeable future. There are also reports that the assays are unable to detect virus in early stages of infection and have given false negatives for up to two weeks. Antibody-based lateral flow immunoassay (LFIA), with short sample to result time, are inexpensive and easy to use and are now coming to market. They are ideally suited for screening, especially in settings with limited healthcare resources such as a clinic or at-home. Companies are developing LFIA to detect the IgM and IgG antibodies produced in patients in response to COVID-19 infection. However, to develop the assays for early detection of infection, one needs: 1) high affinity monoclonal antibodies that are specific to the viral antigen(s) and 2) sufficient sensitivity and specificity to reduce false negative and false positive rates to acceptable clinical limits. Notably, LFIA’s cannot detect SARS-CoV-2 infection in its early stages, which is of great public health concern, and over time the virus can mutate (antigenic-drift) leading to loss of assay performance.

Presently, there are three accepted methods for diagnosis of SARS-CoV-2 infection: (1) viral RNA detection; (2) viral protein detection, typically against the nucleocapsid (N) protein or spike (S) surface glycoprotein; and 3) measurement of specific antibodies directed towards viral proteins. While the initial antibody response may be detected within a week of symptoms (e.g., IgM as early as day 7 and IgG > 14-days post infection), direct viral testing is the preferred screening method in asymptomatic populations and acute presentations. For direct viral testing, reverse transcriptase polymerase chain reaction (RT-PCR) remains the gold standard nucleic acid amplification test (NAAT), with samples collected from nasopharyngeal, mid-turbinate, oropharyngeal (saliva), and bronchoscopy specimens. While RT-PCR tests can provide sensitivity and specificity in ideal settings approaches at about 99%, reported real-world sensitivities are estimated to be as low as 70% for RT-PCR, likely due to variation in sample collection methods and differences in viral shedding across the oropharynx and respiratory tract. Importantly, it is now recognized that presence of RNA detected by PCR may not reflect infectivity, potentially making viral antigen detection a more appropriate biomarker of transmissible disease.

Thus, a key to a broad population screening strategy appears to be to either: (1) significantly increase the throughput of existing PCR-based nucleic acid tests or (2) develop low-cost high sensitivity antigen-based tests that can be rapidly deployed on a large scale without requiring significant infrastructure to be in place. The first lends itself to well-equipped central laboratories; whereas the latter is more amenable to a distributed, POC testing solution.

While there may never be a “perfect test” for SARS CoV-2 (or other highly contagious infectious diseases like it), what is urgently needed is a test with high sensitivity to “rule in,” high specificity to “rule out,” the ability to discern active and past infection, rapid turnaround-time, and a price-point to allow testing at scale. Ideally, such a test could be performed by an inexperienced user (e.g., at-home or in the community), be able to reliably detect early (asymptomatic or acute) infection with a low false positive rate and have results that can be objectively read and easily transmitted to patients’ medical providers and public health personnel.

The disclosed aptamer-based assay platform addresses the aforementioned problems with an aptamer-based electrochemical lateral flow assay for COVID-19 detection that can be read by a simple low-cost glucometer.

Aptamers are single stranded DNA or RNA ligands that are selected through an iterative in vitro process called SELEX (Systematic Evolution of Ligands by Exponential Enrichment). They bind to their protein targets with high affinity and specificity that is analogous to polyclonal and monoclonal antibody binding. Aptamers are easy to develop, inexpensive, thermodynamically stable, and can be used for developing off-the-shelf assays for viral detection. Aptamers with high affinity and specificity have been developed against other viruses, e.g., Anti-gp120 aptamer (HIV-1), anti-HA aptamer (Influenza A) and are currently being developed against SARS-CoV-2 virus. Aptamers can act as a suitable detection probe, owing to their certain advantages compared to counterpart antibody-based systems. Considering the challenges and drawbacks with currently available COVID-19 diagnosis methods, it is thus imperative to develop fast, sensitive, portable, low-cost, and accurate biosensor for COVID-19 detection, specifically from asymptomatic patients.

In some example embodiments described herein, the disclosed aptamer-based antigen detection platform uses aptamers that have been engineered for the receptor binding domain (RBD) of the SARS-CoV2 spike (S) protein, and the nucleocapsid (N) proteins. The virus uses the S protein to bind to the host ACE2 receptor to infect the host’s cells. The N-protein binds to viral RNA and structurally protects the viral genome. It is the most abundant viral protein and is present in high copy numbers in blood and excreted or secreted in urine and saliva, respectively. N-protein can be a biomarker for active COVID-19 infection and the viral load. To minimize the assay and instrumentation cost, the aptamer-sensor includes an analyte conversion process so that the antigen-of-interest (e.g., SARS CoV-2 viral protein(s)) can be indirectly detected via direct detection of another analyte (e.g., glucose) in conjunction with a commercial glucometer for readout.

As illustrated above in FIG. 1B, the disclosed aptamer-based antigen detection platform utilizes aptamers directed at the target antigen, e.g., viral S and/or N protein(s) of COVID 19, which are pre-conjugated to an enzyme through a small antisense oligonucleotide strand that is complementary to a portion of the aptamer’s binding domain (aptatope). The aptamer-antisense complex is conjugated on a substrate capable of receiving the collected patient sample with the target antigen. The enzyme, e.g., invertase, is selected for a second phase of the aptamer-based assay detection technique, where it catalyzes another chemical to a second antigen (e.g., catalyzing sucrose into glucose) that is easily measurable on a low-cost antigen sensor device, such as a glucometer. In some embodiments, the aptamer-antisense complex is configured as a biotinylated aptamer-oligo-invertase complex is pre-assembled on magnetic beads. In the presence of the conjugate antigen, the aptamer undergoes a conformational change, displacing the lower affinity antisense strand, thus creating an antigen sensitive switch. After magnetic separation, the released enzyme invertase hydrolyzes sucrose into glucose, e.g., with a turnover rate of 5×10³ glucose mol/sec, enabling many orders of magnitude signal enhancement. This amplification allows readout with an off-the-shelf glucometer where the signal is proportional to the viral antigen concentration.

In some embodiments, for example, the aptamer-based antigen detection platform is optimized for saliva as the patient sample, e.g., given the simplicity of sample collection. Yet, notably, the disclosed approach works well with other sample types. As discussed below, example implementations of the example SARS CoV-2 virus detection platform demonstrated that the assay has minimal cross-reactivity to proteins from other respiratory viruses, recognizes native antigens in conditioned media of cells infected with SARS-CoV-2, and clinically discriminates infected and non-infected individuals with an unmodified, low-cost glucometer (e.g., purchased for ~$29 USD).

(1) Example materials and methods in the example implementations of the aptamer-based antigen detection platform and technique. Example reagents used in the example implementations included the following. Biotin-tagged, HPLC-grade purified aptamers against SARS-CoV-2 N antigen and SARS-CoV-2 S antigen and complementary thiolated antisense DNA oligonucleotides were custom designed, with pre-modified constituents obtained from Integrated DNA Technology (IDT). The aptamer and antisense sequences used in the example implementations are listed in Table 1. Streptavidin-coated Dynabeads M-280 (2.8 µm), 10% bovine serum albumin (BSA), dithiobis(succinimidyl propionate) (DSP), and tris(2-carboxyethyl) phosphine (TCEP) were obtained from Thermo Fisher. Dulbecco’s potassium phosphate buffer (DPBS) with calcium and magnesium, citrate buffer, calcium chloride (CaCl₂), magnesium chloride (MgCl₂), ethylene diamine tetra acetic acid (EDTA), sodium borohydride (NaBH₄), sucrose, glucose, 4-(N-Maleimidomethyl)cyclohexane-1-carboxylic acid 3-sulfo-N-hydroxysuccinimide ester sodium salt (sulfo-SMCC), glucose oxidase type-VII from Aspergillus niger, and invertase (Grade VII) from Saccharomyces cerevisiae were purchased from Sigma Aldrich. Reagents were analytical grade and used without further processing. Buffer compositions are described in Table 3. SARS-CoV-2 N and S, Influenza A (H1N1) hemagglutinin and neuraminidase, and MERS nucleocapsid and spike RBD fragment proteins were purchased from Sino Biological. Amicon filters (3, 10, and 100 kDa cutoffs) were purchased from Millipore. Dulbecco’s Modified Eagle’s Medium (DMEM) culture media and Penicillin-Streptomycin (10,000 U/mL) antibiotics were obtained from Corning and Gibco, respectively. An “Accu-Chek GuideMe” glucometer was used for all assays.

Conjugation of invertase with the antisense oligomer strand was implemented in the following manner. Invertase was covalently linked with a thiolated antisense oligomer strand (specific to the N or S aptamer). Briefly, 30 µL of 1 mM thiolated antisense oligomer was mixed with 6 µL of 0.5 M TECP and stirred at room temperature (RT) for 2 hours. After incubation, the antisense strand was purified through centrifugation with a 3 kDa cutoff filter. This was repeated 8× in DPBS buffer. Next, 400 µL of invertase was mixed with 1 mg of water-soluble sulfo-SMCC by gentle pipetting for 5 min. The mixture was then placed on a shaker for 2 hours at RT. After incubation, unbound sulfo-SMCC was removed by centrifugation with a 10 kDa cutoff filter. This process was repeated 8× in DPBS buffer. The purified sulfo-SMCC linked invertase (sulfo-SMCC-invertase) was mixed with the purified, reduced, thiolated antisense strand and kept on a shaker for 48 hours at RT. Unreacted free antisense oligomers were removed by centrifugation with a 10 kDa cutoff filter. This was repeated 8× in DPBS. The purified antisense-invertase conjugate was stored at 4° C. for downstream application.

Hybridization of aptamer with antisense-invertase conjugate was implemented in the following manner. The biotinylated (N or S) aptamer was refolded by heat treatment at 80° C. for 3 min, followed by gentle cooling at RT for 5 min. Similarly, the antisense-invertase conjugate was heat treated at 40° C. for 10 min before hybridization. 10 µL of heat-treated N or S aptamer (e.g., 0.5 mM) was mixed with 20 µL of heat-treated antisense-invertase conjugate and 170 µL of DPBS and placed on a shaker for 2 hours at RT. The unhybridized free aptamer was removed by centrifugation with a 100 kDa cutoff filter. This was repeated 8× with washing in DPBS. The purified, hybridized aptamer/antisense-invertase complex (e.g., ~200 µL) was stored at 4° C.

Conjugation of aptamer/antisense-invertase complex and magnetic beads was implemented in the following manner. 200 µL of streptavidin coated magnetic beads (MBs) was placed near a rare earth magnet. The supernatant was discarded and replaced with 600 µL of washing and binding buffer. This process was repeated three times. The MBs were then equilibrated with DPBS buffer for 10 min, the incubation buffer discarded, and resuspended in 200 µL of the biotinylated aptamer/antisense-invertase complex. This was kept on a shaker for 1 hour at RT. Excess unbound aptamer/antisense-invertase complex was washed off with buffer three to five times. The resulting aptamer/antisense-invertase magnetic bead complex (MBC) was treated with 1% BSA in DPBS for 30 min. After incubation, the BSA solution was discarded and the MBC was resuspended in 400 µL of DPBS. 50 µL of the MBC (e.g., ~200 µg) was aliquoted in test tubes and stored at 4° C.

Fabrication of the custom electrochemical glucose sensor was implemented in the following manner. A glass slide with an evaporated gold electrode (e.g., 5 nm Ti / 50 nm Au) was chemically cleaned in piranha solution (e.g., 3:1 of H₂SO₄:H₂O₂) for 1 min followed by washing with ultrapure (milli-Q) water. The electrode was then sonicated in acetone and isopropanol sequentially for 5 min followed by washing with ultrapure water. The electrode was then electrochemically cleaned in 0.5 M H₂SO₄ by sweeping the potential from -0.5 to +1.2 V vs. Ag/AgCl electrode, washed with water, and air dried. A surface assembled monolayer (SAM) was formed by incubating the electrode in 1 mL DSP (e.g., 2 mg/mL) reduced with 5 µL of (e.g., 10 mg/mL) NaBH₄ for 2 hours at RT. The electrode was then washed with acetone, methanol, isopropanol, and ultrapure water followed by air drying. The DSP modified electrode was incubated with 5 µM of glucose oxidase in PBS overnight at 4° C. to covalently link to the DSP modified surface. The unbound GOx was washed off with PBS and the sensor was incubated in 1% ethanol amine for 15 min to block any remaining active succinimidyl and then 1% BSA for 10 min. The electrode was stored at 4° C. when not in use. Layer by layer assembly was monitored by cyclic voltammetry (CV) with a CHI-760E electrochemical workstation in a three-electrode configuration (BASi Ag/AgCl reference electrode and a platinum wire counter electrode). Voltammograms were measured from -0.5 to +0.8 V at a scan rate of 50 mV/s with 1 mM ferrocene in 1×PBS and 0.25 M KCl.

Custom-made glucose sensor SARS-CoV-2 assay was implemented in the following manner. 100 µL of DPBS buffer spiked with SARS-CoV-2 N or S protein was incubated with 200 µg of MBC (N or S) with gentle shaking for 30 min at RT in a 1.5 mL centrifuge tube. The MBC was pulled down using a rare earth magnet. 90 µL of the supernatant was transferred to another centrifuge tube prefilled with 100 µL of 2× Measurement buffer. After incubating for 30 min, 200 µL was placed on the glucose sensor and readout using a CHI-760E electrochemical workstation with the three-electrode configuration described previously. Voltammograms were measured from -0.5 to +0.8 V at a scan rate of 50 mV/s.

Collection of nasopharyngeal swab and saliva samples was implemented in the following manner. Matched nasopharyngeal swabs (NPS) were collected in RNA Shield DNA/RNA storage medium (Zymo) or viral transport medium and saliva samples (no additive) from symptomatic and asymptomatic study subjects with a prior positive clinical COVID-19 RT-qPCR result under institutional review board (IRB) approval. Informed consent was provided from all subjects. These samples were subjected to viral RNA extraction using the MagMax Viral/Pathogen Nucleic Acid Isolation Kit (Thermo) and the TaqPath COVID-19 multiplex RT-qPCR assay was performed on the resulting RNA samples. Saliva in 300 µL aliquots were provided as blinded specimens for testing in the BSL-3 lab and all experiments were performed in accordance with the relevant guidelines and regulations designated by the UCSD Human Research Protections Program (HRPP).

Glucometer-based SARS-CoV-2 assay was implemented in the following manner. Saliva samples (e.g., ~300-500 µL) were collected in sterile tubes from volunteers using a standard passive drooling technique. To test N protein, saliva was mixed with 1% Triton, whereas no detergent was added to samples where protein S was to be detected. 100 µL of sample (e.g., contrived, conditioned media, or saliva) was then diluted two-fold in DPBS buffer and assayed without any further processing or treatment. Half of the diluted sample was then incubated with 200 µg of MBC (N or S) with gentle shaking for 30 min at RT in a 1.5 mL centrifuge tube. The MBC was pulled down using a rare earth magnet. 90 µL of supernatant was transferred to a centrifuge tube prefilled with the Sucrose buffer. The other half of the diluted sample was placed in a separate centrifuge tube prefilled with the Sucrose buffer as a background control. After mixing, the tubes were incubated at 60° C. in a water bath for 1 hour. Finally, 10 µL of each reaction solution was placed on a glucometer test strip and read out using a glucometer. The difference between the two readings was recorded. Measurements were repeated in triplicates.

SARS-CoV-2 production and quantification was implemented in the following manner. Vero E6 cells were obtained from ATCC and grown in DMEM with 10% fetal bovine serum (FBS) and Penicillin-Streptomycin. SARS-CoV-2 isolate USA-WA1/2020 (BEI Resources) was propagated and aliquots of secreted virus in culture media were stored at -80° C. Infectious units (IU) were quantified by digital droplet PCR (ddPCR) and plaque assay using Vero E6 cells. For ddPCR, viral stock media was added to TRIzol LS (ThermoFisher) and RNA extracted using a Directzol RNA miniprep kit (Zymo Research). The ddPCR quantified SARS-CoV-2 ORF1a was performed. For plaque assay quantification, viral supernatants were 10-fold serially diluted in DMEM without serum. Vero E6 cells in 12-well plates were washed with PBS, and 200 µL of virus dilution was added per well and incubated 1 hour at 37° C. with rocking every 10-15 min. The inoculum was removed and 1 mL of overlay (0.6% agarose in MEM with 4% FBS) was added to each well. Overlays were prepared by mixing equal volumes of 1.2% agarose and 2× MEM supplemented with 8% FBS, 2× L-glutamine, 2× non-essential amino acids, and 2× sodium bicarbonate. Assays were incubated for 48 hours at 37° C. and fixed by adding 2 mL 10% formaldehyde for at least 24 hours. Overlays were removed, and monolayers were stained with 0.025% crystal violet in 2% EtOH and plaques counted.

(2) Example implementations of an antisense release oligo for anti-S and anti-N aptamer, e.g., via testing the release of antisense oligo from the aptamer upon antigen (S and N protein) binding by PCR. To test the release of the antisense oligo from the aptamer upon ligand binding, a validating PCR-based assay was designed.

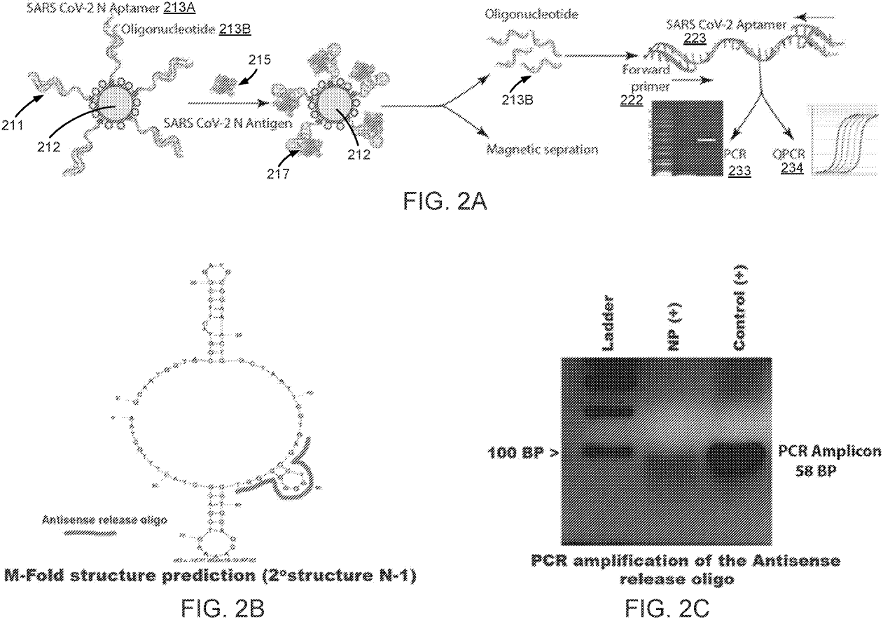

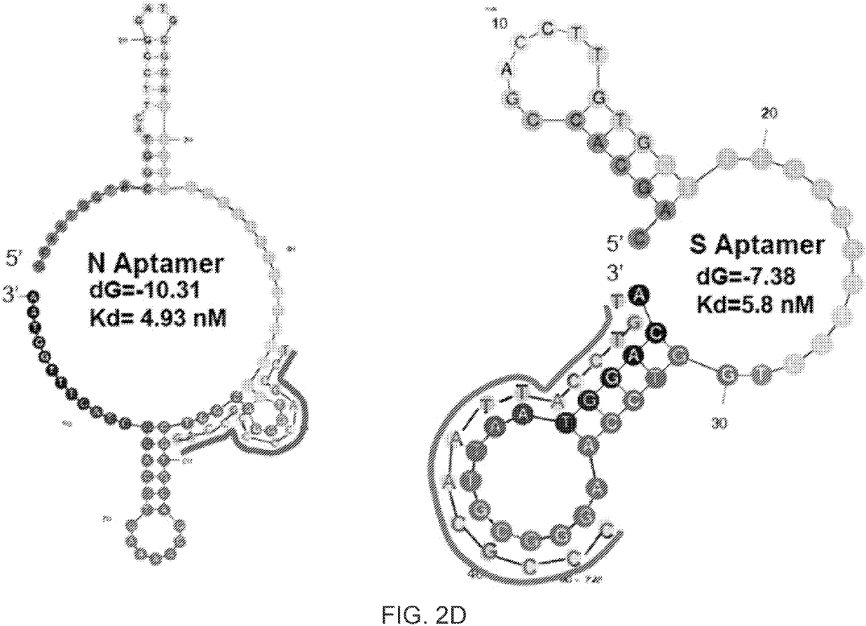

FIGS. 2A-2E show an illustrative diagram and example data of an example PCR-based assay validation method to test the release of an antisense oligo upon a target antigen binding to an example aptamer sensor, in accordance with the present technology. The example PCR-based assay validation method served as a competitive aptamer validation assay, demonstrating the capability of the disclosed technology for release of engineered antisense strand with structure switching of the engineered aptamer in presence of the target antigen. FIGS. 2A and 2C provide an illustrative schematic of this method and example confirmation PCR data. In this example, the secondary structure of the anti-N protein aptamer for SARS-CoV-2 N protein is shown (e.g., generated from M-fold software), where the red line indicates the binding site for the antisense oligo strand.

FIG. 2A shows an example aptamer-oligo complex 214, comprising a SARS CoV-2 anti-N aptamer 213A bound to a complementary oligonucleotide 213B (i.e., antisense oligo), immobilized on magnetic bead 212, which is exposed to SARS CoV-2 N protein antigen 215, where protein binding of the SARS CoV-2 N protein antigen 215 to the SARS CoV-2 anti-N aptamer 213A causes release of the antisense oligo 213B. Notably, the example aptamer-oligo complex 214 can include the SARS Cov-2 S aptamer bound to a respective complementary oligonucleotide. In implementations of the example PCT-based assay validation method, after magnetic separation of the SARS CoV-2 N protein antigen-aptamer conjugate 217 bound to the magnetic bead 212, the released antisense oligo 213B was collected from the supernatant of the solution (e.g., after implementation of the magnetic separation), in which the antisense oligo was added to a PCR reaction mixture 219 with the SARS Cov-2 aptamer 223 (e.g., N aptamer in the diagram of FIG. 2A) as the template and a forward primer 222 for characterization in a PCR reaction. The PCR reaction was resolved in the agarose gel and stained with Ethidium Bromide (EtBr). The PCR amplification was confirmed through agarose gel electrophoresis or quantitative QPCR, illustrated by sample PCR plot 233 and sample QPCR plot 234, respectively.

High-affinity DNA aptamers were selected against the S and N proteins (SARS CoV-2 antigens). The sequences of these aptamers were analyzed using M-fold to determine the secondary structures, as shown for the N-aptamer in FIG. 2B and both the N-aptamer and S-aptamer in FIG. 2D. Each of the antisense oligos (shown in red highlight) that initially binds to the N-aptamer and the S-aptamer is also shown in FIG. 2D. In the absence of the target antigen (S and N protein), the antisense oligos anneals to the aptamers through Watson-Crick base pairing. However, in the presence of the target, the aptamers undergo a conformational rearrangement, resulting in the release of the oligo.

Thus, the engineered antisense oligo and anti-S and anti-N protein aptamers provide an antigen sensitive switch that readily displaces the antisense oligo due to the higher binding affinity (10³×) of the target protein. The example implementations confirmed the oligo release through these initial experiments. Example data for the N-aptamer is shown in the FIG. 2C and in FIG. 2E.

FIG. 2C shows a PCR gel image depicting release of antisense strand (released from the example anti-N aptamer) in the presence of the target N protein. Notably, the S-aptamer was tested in the same fashion. These example results established the experimental conditions for the subsequent enzymatic assay.

FIG. 2D show diagrams of the predicted secondary structure of the N-aptamer (designated by label 281) and the S-aptamer (designated by label 282), using M-fold software. In the diagrams 281 and 282 corresponding to the secondary structure of the N-aptamer and the S-aptamer, the antisense strand sequence and the binding locations are annotated in red.

Table 1 shows the sequences of the example N-aptamer (anti-N protein aptamer) and S-aptamer (anti-S protein aptamer), in which the N-aptamer and S-aptamer are bound to Biotin at a terminus of the sequence. Table 1 also shows the sequences of the example antisense strands.

TABLE 1

| | | |
|---|---|---|
| Aptamer Target | Aptamer Sequence (5′-3′) | Antisense Sequence (5′-3′) |
| SARS-CoV-2 N | Biotin/TTTTTTGCAATGGTACGGTACTTCCGGA TGCGGAAACTGGCTAATTGGTGAGGCTGGGG CGGTCGTGCAGCAAAAGTGCACGCTACTTTGC TAA | Thiol/TTTTTTTTTT TTGACCGCCCCA GCCT |
| SARS-CoV-2 S | Biotin/TTTTTTCAGCACCGACCTTGTGCTTTGG GAGTGCTGGTCCAAGGGCGTTAATGGACA | Thiol/TTTTTTTTTT TTTGT CCATTAACGCCC |

FIG. 2E shows PCR gel image plots depicting release of the antisense strand (AS) released from the example anti-N aptamer (left plot) in the presence of the target N protein and the antisense strand (AS) released from the example anti-S aptamer (right plot) in the presence of the target S protein. The example antisense release study from the hybridized N and S aptamer immobilized on the magnetic beads upon antigen binding was confirmed with PCR. In this example study, the PCR products were resolved in 2% agarose gel and stained with EtBr for visualization of the DNA amplicon. PCR reactions with the respective aptamer templates, forward and reverse primers was performed for (+) control. For the (-) control, only buffer (without N or S proteins) was added to the MB-aptamer-antisense (AS) complex. A 100 base-pair ladder was also resolved as molecular-weight marker.

Table 2 shows the sequences of the example PCR primers used in the example implementations of the PCR-based assay validation method.

TABLE 2

| | | |
|---|---|---|
| Aptamer Target | Forward Primer (5′-3′) | Antisense Sequence (5′-3′) |
| SARS-CoV-2 N | GCAATGGTACGGTAC | GACCGCCCCAGCCT |
| SARS-CoV-2 S | CAGCACCGACCTTG | TGTCCATTAACGCCC |

The example implementations included the following techniques for the conjugation of antisense oligo to the invertase enzyme. For example, the 5′ thiol-antisense oligo and the invertase enzyme (B-fructofuranosidase) were conjugated using sulfosuccinimidyl-4-(N-maleimidomethyl) cyclohexane-1-carboxylate (sulfo-SMCC) as linkers. The unconjugated 5′ thiol-antisense oligo was removed from the reaction mixture using a spin column with a 100 kDa molecular-weight cutoff membrane (Amicon-100K). The oligo-invertase conjugates were analyzed using a standard electrophoretic mobility shift assay (EMSA). 4-20% gradient PAGE gel was stained with cyber gold and Coomassie blue for visualization of DNA and protein, respectively.

Table 3 shows the buffer composition used for various reactions in the example implementations.

TABLE 3

| | |
|---|---|
| Name | Composition |
| Washing and binding buffer | 10 mM tris, 0.5 M NaCl, 1 mM EDTA, pH 7.4 |
| Sucrose buffer (4X) | 0.4 M citrate, 4 M sucrose, 20 mM CaCl2, 4 mM MgCl2, 2 mM EDTA, 11.1 mM glucose, pH 5.0 |
| Measurement buffer (2X) | 2 M sucrose, 2 mM ferrocene, 10 mM CaCl2, 2 mM MgCl2, 1 mM EDTA in PBS pH 7.4 |

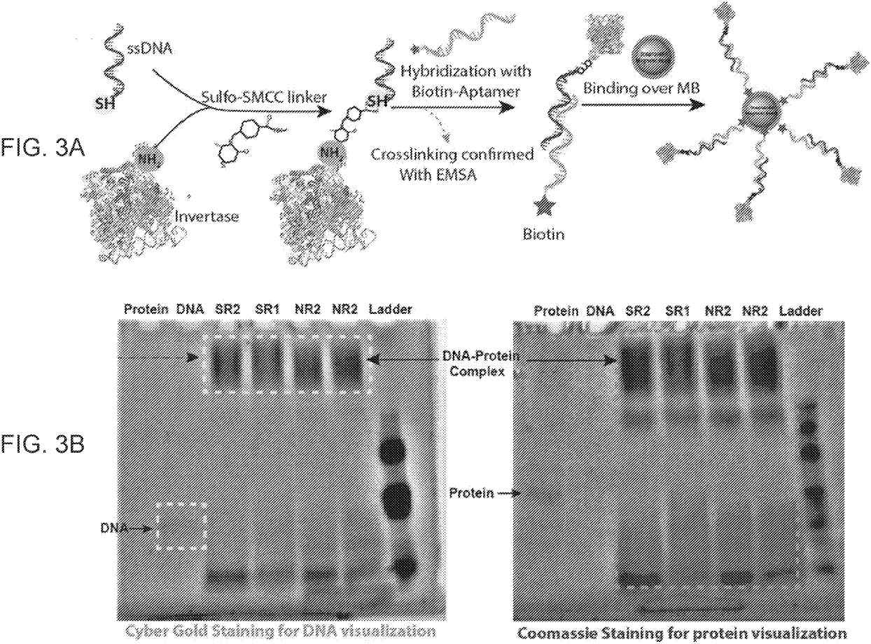

FIGS. 3A and 3B show an illustrative diagram depicting an example conjugation technique and example data plots from electrophoretic mobility shift assay. FIG. 3A shows an illustrative diagram depicting an example conjugation technique for an example embodiment of the invertase-antisense oligo-aptamer-magnetic bead complex. FIG. 3B shows example data plots from electrophoretic mobility shift assay. N-antisense (N-AS) and S-antisense (S-AS) conjugation with invertase was verified by an electrophoretic mobility shift assay (EMSA). Unconjugated DNA and the invertase protein were run as controls. S-AS1, S-AS2 and N-AS1, N-AS2 depict two different concentrations of antisense-invertase enzyme conjugates. The gel was stained with Cyber Gold, followed by Comassie brilliant blue for DNA and protein staining, respectively. Higher migrating bands were detected at the same spot with both the DNA and protein specific dyes, thus indicating successful conjugation. As depicted in FIG. 3B, the DNA-protein complex was visible at the same spot, e.g., indicating the efficacious crosslinking between antisense oligo and invertase.

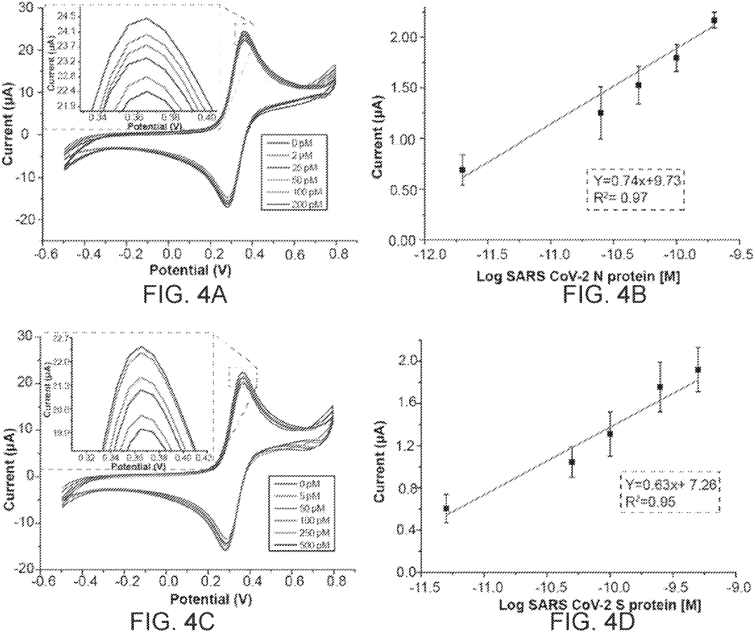

(3) Example implementations of COVID-19 N/S protein detection using a benchtop potentiostat. In some example experimental implementations, prior to testing with commercial glucose test strips, a benchtop potentiostat was used, which was custom-made electrodes functionalized with glucose oxidase and an electron transfer mediator, ferrocene. The developed sensors were exposed to the supernatant solutions released after incubation with various concentration of spiked SARS-CoV-2 N/S antigen. The displaced antisense-invertase conjugate concentration was directly correlated with the antigen concentration, as shown in FIG. 4A and FIG. 4C by the increase in the oxidation peak in the voltammogram. There was a linear dependence on concentration over the range of 2-200 pM (FIG. 4B) for the N protein and 5-500 pM (FIG. 4D) for the S-protein. The calculated limit of detection (LOD) is 1.53 pM and 0.87 pM, for the N and S protein, respectively.

FIGS. 4A-4D show data plots depicting example results of an example implementation for detecting N and S protein using the example assay 100′, using a benchtop potentiostat. FIGS. 4A and 4C show voltammograms of various concentrations of SARS-CoV-2 N and S protein in PBS, respectively. FIGS. 4B and 4D shows calibration curve showing signal versus log N and S protein, respectively. The measurements were done in triplicates and error bars represent ±1σ.

(4) Example implementations of COVID-19 N/S protein detection using a glucometer. The same procedure was repeated using commercial off-the-shelf glucose test strips and a glucometer. After spiking protein at various concentrations and removing the magnetic beads, the supernatant was incubated with 2 M sucrose at room temperature for ~30 min. The resulting glucose was measured on an Accu-Chek Guide Me glucometer purchased at a drug store for $29.

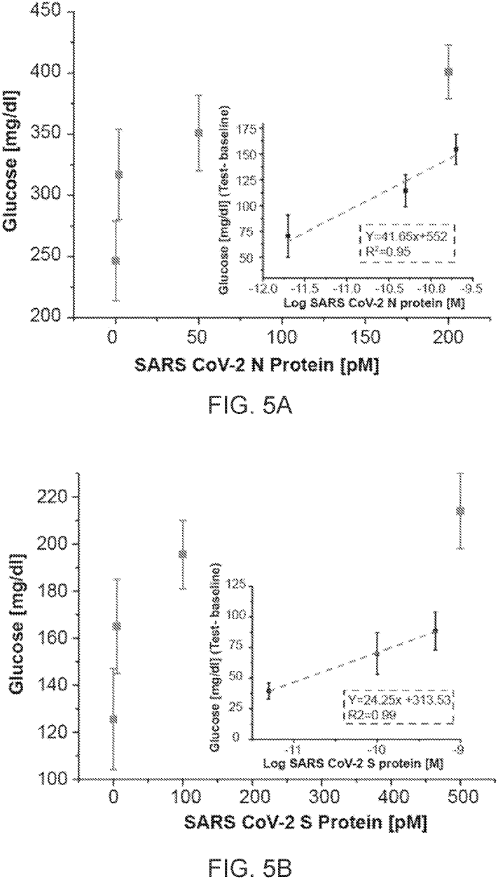

FIGS. 5A and 5B shows data plots depicting example results of an example implementation for detecting N and S protein using a glucometer. FIG. 5A shows the glucose reading plotted versus the N protein antigen concentration along with a calibration curve that subtracts off the blank; and FIG. 5B shows the glucose reading plotted versus the S protein antigen concentration along with a calibration curve that subtracts off the blank. In this example, the sensitivity of the assay was found to be 41.65 [glucose mg/dL]/log[SARS-CoV-2 N protein] pM and 24.25 [glucose mg/dL]/log[SARS-CoV-2 S protein] pM. This translates into an LOD of 2.34 pM and 2.64 pM for the N and S protein, respectively, with a linear dynamic range of 2-200 pM and 5-500 pM for the N and S protein, respectively.

As shown in FIG. 5A, the N protein spiked into PBS at three concentrations (e.g., 2, 50, and 200 pM) and was read out on an example glucometer. The example inset in FIG. 5A shows background subtracted calibration curve. As shown in FIG. 5B, the S protein spiked into PBS at three concentrations (e.g., 5, 100, and 500 pM) and was readout on the example glucometer. The example inset in FIG. 5B shows background subtracted calibration curve. The measurements were done in triplicates and error bars represent ±1 σ.

(5) Example implementations for saliva detection using a glucometer. The specificity and functionality of example assay was also analyzed in biological fluid. The SARS CoV-2 virus N and S proteins were spiked at various concentration in human saliva 2-fold diluted with PBS to mimic the above sample process.

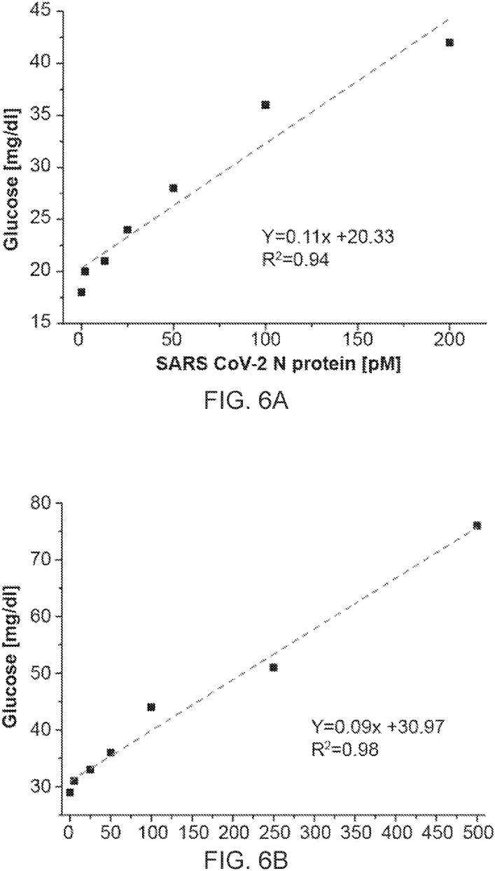

FIGS. 6A and 6B show data plots depicting example results from example implementations for detection of N and S protein in saliva using a glucometer. FIG. 6A shows a glucose to N protein calibration curve; and FIG. 6B shows a glucose to S protein calibration curve. The glucometer readings shown in FIGS. 6A and 6B demonstrate a strong correlation with the glucose concentration with a linear detection range of 2-200 pM and 5-500 pM for SARS CoV-2 N and S proteins, respectively. As shown by the example data, the glucometer-based assay offers the capability of SARS CoV-2 N or S antigen detection with a wider detection range at low cost, e.g., as compared to existing ELISA methods, which offer a detection range of 2.1-128 pM, 21-377 pM for N and S protein respectively for detection of COVID-19.

As shown above, the example studies demonstrated the capability of using the disclosed aptamer-based SARS CoV-2 assay method to detect N and S proteins through an example embodiment of the POC aptamer-based viral assay, which can utilize a commercially available glucometer to diagnose COVID-19 through an example apta-sensor switch. In the example implementations, the sensor achieved an LOD and a dynamic range of 2-200 pM and 5-500 pM for SARS CoV-2 N and S protein in saliva, respectively. For comparison, commercial ELISA kits have a detection range of 2.1-128 pM and 21-377 pM for the N and S protein, respectively.

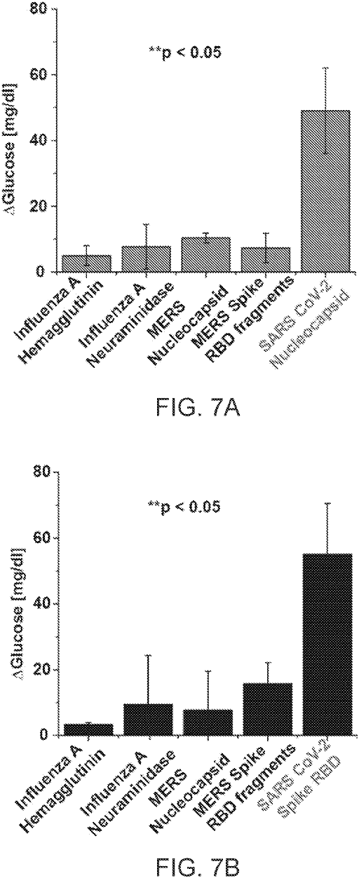

(6) Example implementations for characterizing the specificity of the assay. As a preliminary assessment of assay specificity, signal generation was measured in the presence of antigen from non-SARS-CoV-2 respiratory viruses, namely: Influenza A (H1N1) and MERS-CoV. The SARS CoV-2 virus N protein and S protein specific aptamer complexes were assayed with the off-target antigens at a fixed concentration of 500 pM with the conditions described above. This specificity assay was performed in buffer rather than saliva to isolate the source of the non-specific binding. The example results, shown in FIGS. 7A and 7B, indicate minimal cross-reactivity in the assay, even when the off-target antigens are present in high concentration. As expected, for example, the SARS-CoV-2 N aptamer displayed the highest signal with the MERS nucleocapsid antigen, and the S aptamer with the MERS-CoV RBD antigen, consistent with reported homology between the two coronavirus genomes. In these example implementations, for example, the corresponding SARS-CoV-2 signal was >300% higher than the off-target recording (p < 0.05).

FIGS. 7A and 7B show data plots depicting example data from a cross-reactivity study. The specificity assays used analogous proteins with respect to the example N aptamer complex (shown in FIG. 7A) and example S aptamer complex (shown in FIG. 7B). The proteins were spiked into DPBS at 500 pM. The measurements taken with n=3 and error bars represent ±1 σ.

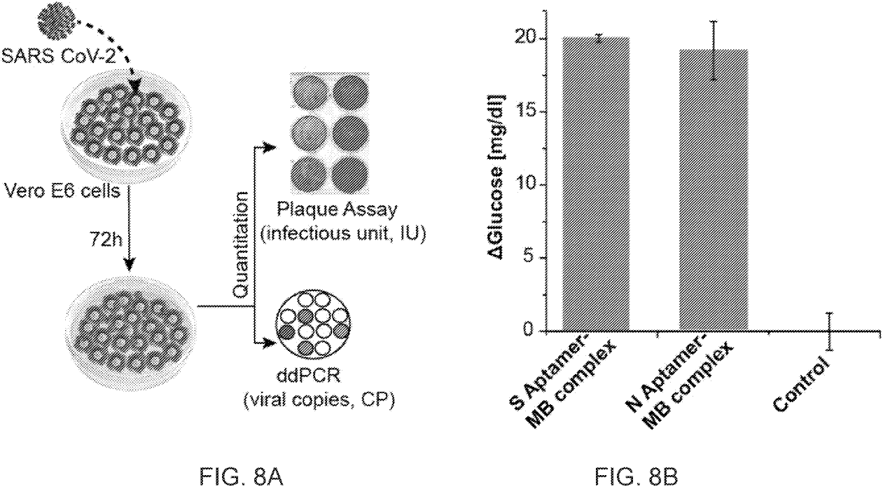

(7) Example implementations for characterizing actual SARS-CoV-2 N and S proteins in cultured media. The previous example studies of SARS CoV-2 N and S protein directed aptamers validated binding with recombinant purified proteins. In some example implementations, to evaluate the example S and N aptamer/antisense-invertase system for recognizing authentic virus and native proteins produced during SARS-CoV-2 infection, a viral stock of SARS-CoV-2 was created and validated in a biosafety level 3 (BSL-3) laboratory. For example, inoculated authentic SARS-CoV-2 were isolated USA-WA1/2020 onto Vero E6 cells and allowed to propagate and secrete virus into the supernatant (FIG. 8A). Supernatant was collected, aliquoted, and frozen. The quantity of SARS-CoV-2 in these preparations was determined by two methods: (1) the quantity of SARS-CoV-2 ORF1a RNA was measured using ddPCR, and (2) the number of infectious virions was determined by plaque assay. SARS-CoV-2 supernatants were diluted 1:10 with DPBS to 52×10⁶ copies and 12.5×10³ IU and assayed with the S and N aptamer complex. The cell media used to propagate the virus has a high level of glucose (450 mg/dL). To nullify the effect of background glucose in the conditioned media, glucometer readings were conducted with control diluted media in a similar manner, but in the absence of the aptamer complexes. As shown in FIG. 4B, both the N and S aptamers showed significant increases in glucometer readings compared to control samples. The example results of this set of example implementations demonstrates that the aptamer/antisense-invertase systems recognize their native targets when produced by replicating authentic SARS-CoV-2.

FIGS. 8A and 8B show an illustration and data plots depicting example data from example implementations for detection of protein N and S using authentic SARS-CoV-2. FIG. 8A shows an illustrative schematic of authentic SARS-CoV-2 virus preparation and quantification of viral RNA by ddPCR and infectious units by plaque assay. FIG. 8B shows example data depicting N and S aptamer/antisense MB complex detection of the SARS-CoV-2 native protein in 1:10 diluted virus culture media. The background media (control) values were subtracted from the measurement results; measurements were taken with n=3; and error bars represent ±1 σ.

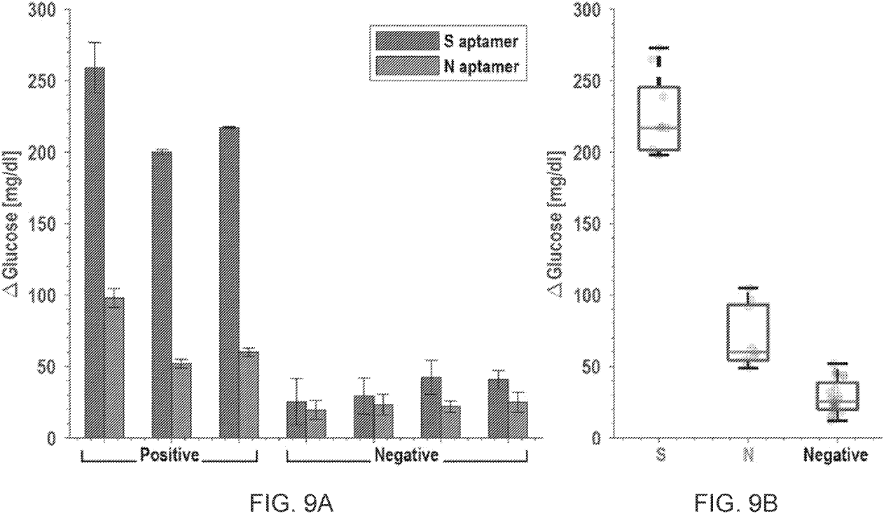

(8) Example implementations for detection of SARS-CoV-2 in clinical specimens. Further example implementations of the example SARS CoV-2 N and S protein aptamer-based assay evaluated whether the developed assays could discriminate between SARS-CoV-2-infected individuals and healthy individuals with validated saliva samples. An example study used a small cohort of three infected persons (e.g., confirmed positive with RT-qPCR) and four healthy controls tested for N protein and S protein binding. The results, shown in FIGS. 9A and 9B, demonstrate the ability of both assays to correctly differentiate between infected and non-infected individuals. Notably, in these example results, the S protein assay showed significantly higher signal-to-control than the N protein assay. In this cohort of 24 individuals, 42% were female and the average age was 31 years. Of the 16 infected individuals, the average time between symptom onset and testing was 7 days, 63% had a fever, and 50% had cough. Two subjects self-reported having asthma, one was pregnant, and none were diabetic.

FIGS. 9A and 9B show example data plots depicting COVID-19 clinical saliva samples. FIG. 9A shows a plot depicting measured data from confirmed positive patients (n=3; patients 23, 30, and 42) and healthy volunteers (n=4) for paired N and S aptamers. Detection of SARS CoV-2 N protein was performed with the addition of 1% Triton to ensure the release of the nucleocapsid protein. Error bars represent ±1 σ. FIG. 9B shows an box and whisker plot.

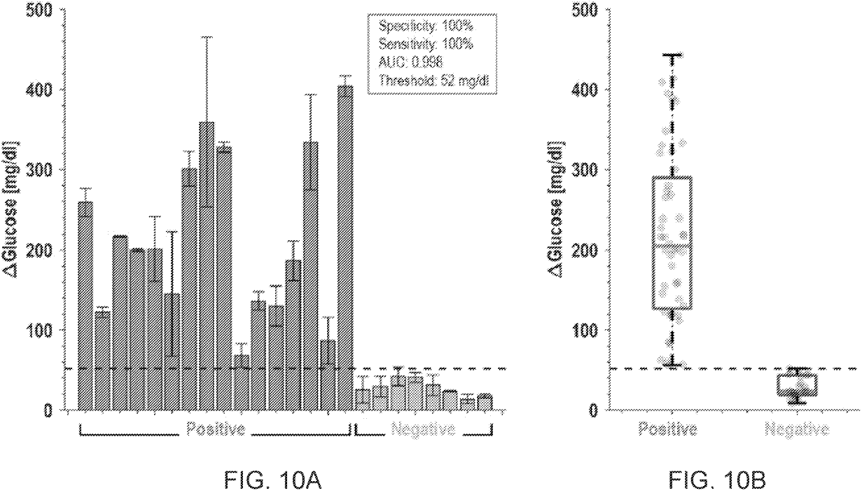

FIGS. 10A and 10B show data plots depicting example results from clinical performance of the example assay using saliva samples from COVID-19 patients and healthy volunteers. The results were presented as a blind panel run under BSL-3 conditions over the course of two days using the same glucometer and a single lot of commercial test strips. The SARS-CoV-2 confirmed positive samples demonstrated higher glucose production (µ = 218 mg/dL, range = 68-404 mg/dL) than healthy individuals (µ = 24 mg/dL, range = 14-37 mg/dL). Receiver operator curve (ROC) analysis yielded an ideal cutoff of 52 mg/dL, which classified positive and negative samples with a sensitivity and specificity of 100% (AUC = 0.9988). The example data has 100% positive percent agreement (PPA) and 100% negative percent agreement (NPA) with the RT-qPCR data performed on the same samples.

Selection of SARS-CoV-2 anti-S and anti-N protein aptamer(s). The disclosed aptamer-based antigen detection platform allows for the selection of novel aptamers against SARS-CoV-2 (S and N) proteins using native protein, Virus Like Particles (VLPs), and/or inactivated virus with preserved antigenicity. These can be assessed for affinity for cultured virus. Briefly, for example, a 2′ Fluoro-pyrimidine modified RNA library can be synthesized. This modified RNA is more stable than DNA or unmodified RNA aptamers against nucleases present in bodily fluids like saliva. The modified RNA aptamer library can be incubated with biotinylated S and N recombinant proteins. Protein-bound RNA oligos can be partitioned from the unbound oligos using an agarose-streptavidin column. After extensive washing, the RNA can be eluted from the protein and amplified using RT-PCR to generate a pool of oligos that are enriched for binding to the target protein. The pool can be used for the next round of selection. Nine to ten, for example, such rounds can be performed with increasing stringency to select aptamers with high affinity against the S and N proteins. Further, negative selection steps using S and N proteins can be incorporated from the closely related SARS-CoV. This can remove (deselect) aptamers that cross-react with other SARS coronavirus, increasing the specificity against SARS-CoV-2.

Technology scale up. Since the example assay described here is nucleic acid based (e.g., no requirement for culturing to produce antibodies), and the other reagents are readily available, it is possible to mass produce quickly with the ability to reach global scale. There are already tens of millions of glucometers on the market today and hundreds of millions could be produced quickly. Thus, it is envisioned that the example POC SARS CoV-2 virus detection method and system can provide reliable “sample-to-answer” in <20 minutes that can be scaled with our commercial partner. Notably, the semi-quantitative nature of this assay may provide further public benefit if the science shows relationships between antigen load and infectivity or is predictive or more severe illness and worse outcomes.

Example Cost Summary. With low volume production, it is estimated that some example embodiments of the example POC antigen detection platform would cost $3.00/test (e.g., estimated from $0.20/test strip, $0.50/aptamer, $0.20/invertase (100 units), $2.0 /100 µg magnetic beads, and $0.10/reagents). Yet, notably, the example POC antigen detection platform can be scaled to higher volume production, thereby reducing the estimated costs to less than $1/test with large-scale production. This is inexpensive enough that it could routinely be performed at home by anyone - including students as part of the broad testing plan needed to reopen public places such as airports, university campuses, places of business, etc. Glucometers are already manufactured at scale and readily available for purchase for less than $50 USD.

Example Comparison. To situate the example POC antigen detection assay for SARS CoV-2 in the landscape of other available SARS-CoV-2 diagnostics, performance data was compared.

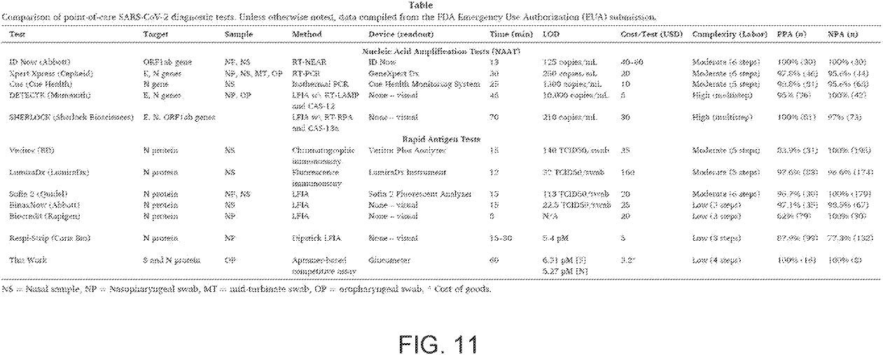

FIG. 11 provides a summary of reported point-of-care diagnostic tests. In the table shown in FIG. 11, a diversity of approaches is shown that span across the spectrum of nucleic acid and antigen modalities. However, there are notable gaps, including a paucity of other saliva-based antigen tests and a complete lack of tests that provide a quantitative readout. The example POC aptamer-based COVID-19 detection assay, as shown by the example results discussed above, demonstrates that it fills this gap without sacrificing the sensitivity or specificity, and while being capable of using devices that already exist at scale with an easily acquired sample.

### SARS-CoV-2 Assay Using a Custom Analyte Reader

In some embodiments, the disclosed aptamer-based viral assay can be used to detect a target antigen of a virus through the disclosed antigen-switch detection technology using a custom analyte reader, e.g., which can obviate the need for a standard glucometer.

In some example embodiments, for example, the custom analyte reader can include a potentiostat-based glucose detection device that can measure glucose concentrations in inputted samples, and thereby de-risk the use of an off-the-shelf glucometer. In some embodiments, the custom analyte reader can include a customized Bluetooth-communicative potentiostat electronics device to implement (automate) the assay and wirelessly transfer the assay data to a mobile device, which can include a customized software application (app) associated with the disclosed aptamer-based viral assay.

Example embodiments of the custom analyte reader are shown below, including a first embodiment comprising a miniaturized electrochemical sensor device couplable to a receptacle (or receiving zone) of an assay housing, and a second embodiment comprising a hand-held electrochemical sensor device having a receiving body that couples to an assay housing, from which the solution containing the released enzyme transfers to a chamber of the hand-held device having an electrochemical sensor for analyzing a corresponding substance that correlates with a parameter of the virus antigen.

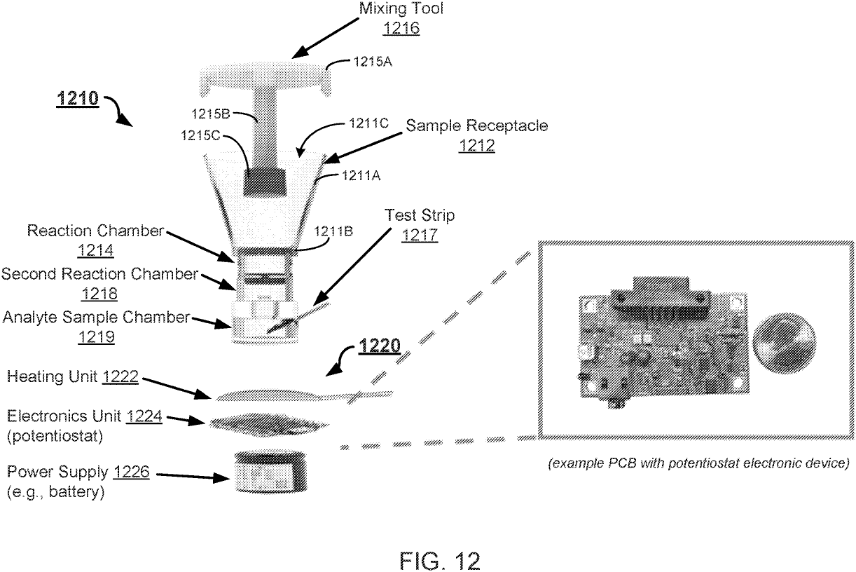

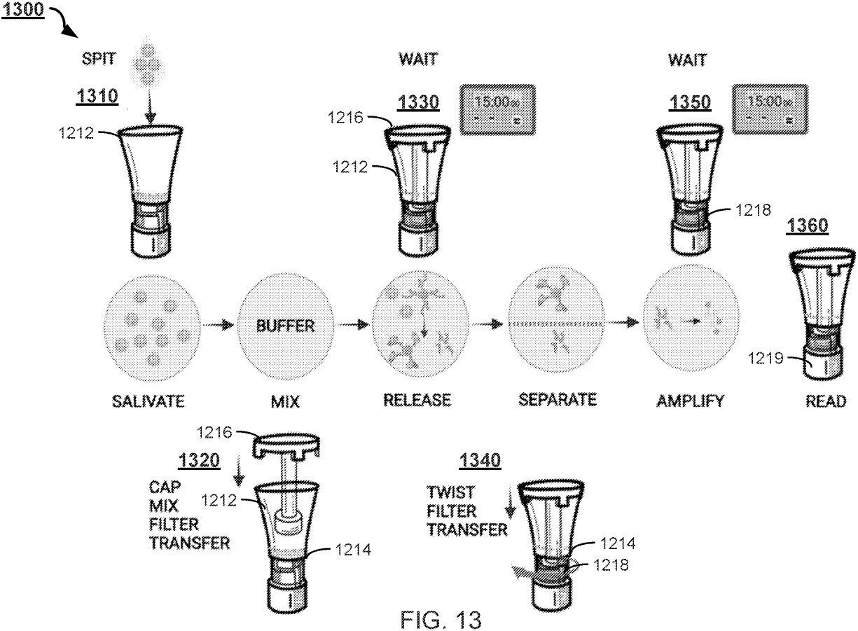

In some embodiments, the disclosed viral assay platform includes a low-cost electronic module capable of quickly heating the sample to a fixed temperature for a set period (e.g., 30 or 60° C. for 15 minutes). As an alternative strategy to the use of over-the-counter glucometers, for example, a custom potentiostat device is integrate with a power and heating stack that can be utilized in implementations of the molecular assay, as illustrated in FIGS. 12 and 13.

FIG. 12 shows a diagram illustrating an example embodiment of a semi-automated test container (SATC) 1210 couplable to an example embodiment of a custom analyte meter 1220, in accordance with the present technology.

The SATC 1210 includes a sample receptacle 1212 comprising a container having one or more sidewalls 1211A and a bottom wall 1211B with an opening 1211C to allow a patient to provide a biological sample (e.g., saliva sample, by the patient spitting in the sample receptacle 1212 to a predetermined level). The SATC 1210 includes a reaction chamber 1214, which is disposed below the sample receptacle 1212, and which can be configured to include the aptamer-based assay device, such as the example embodiment including a substrate (e.g., magnetic or polystyrene particles) conjugated with one or more aptamers (e.g., the anti-S and/or anti-N protein aptamers) initially bound to an enzyme-tagged oligonucleotide having a complementary strand with an affinity to the one or more aptamers). For example, the reaction chamber 1214 can include a solution (e.g., one or more buffers) that contains the aptamer-based assay device. In some embodiments, for example, the SATC 1210 can include a mixing tool 1216 comprising a handle 1215A from which a shaft 1215B projects, which can optionally include an end 1215C having a larger size than the shaft 1215B and/or fins to (i) cause the patient sample to enter the reaction chamber 1214 and/or (ii) cause the churning or mixing of the patent sample with the solution containing the aptamer-based assay device in the reaction chamber 1214. The SATC 1210 includes a second reaction chamber 1218, which is disposed below the reaction chamber 1214, to facilitate conversion of a substance that is to convert to the analyte for measurement based on the enzyme released during the reaction in the reaction chamber 1214. In implementations, for example, the patient sample containing the viral antigen reacts with the aptamer-based viral assay device to bind to the one or more aptamers of the device while releasing the enzyme-tagged complementary strands into the solution. The substrate (e.g., polystyrene or magnetic particles) are separated from the released enzyme-tagged oligonucleotides, which are to be separated into the second reaction chamber 1218. In some embodiments, the second reaction chamber 1218 can contain a second solution (e.g., one or more buffers) containing a primary substance that is to be converted into the analyte measurable by the custom analyte reader 1220. The converted analyte can be passed into an analyte sample chamber 1219 of the SATC 1210, which can be collected for testing by the custom analyte meter 1220.

The custom analyte meter 1220 includes a heating unit 1222, including a heater, configured to couple to a portion of the SATC 1210 where a processed sample containing the released enzyme is collected. In some embodiments, the heater of the heating unit 1222 can include a Peltier element with a temperature sensor, e.g., for closed-loop control by a data processing unit of the custom analyte meter 1220. The custom analyte meter 1220 includes an electronic unit 1224 that includes a printed circuit board (PCB) having an electrical circuit including a potentiostat configured to control an electrochemical sensor for electroanalysis of a solution containing a converted analyte that was converted from a primary substance into the converted analyte via the released enzyme from the example aptamer-based assay. The custom analyte meter 1220 includes a power supply 1226, which is in electrical communication with the electronic unit 1224 and/or the heating unit 1222. As shown in examples herein, the converted analyte can include glucose that is detected by the potentiostat, where the enzyme released during implementation of the example aptamer-based assay can include invertase, and the primary substance that is catalyzed by the invertase is sucrose to be converted to the glucose analyte.

In some implementations of the example custom analyte meter 1220, a test strip 1217 having a solution containing the analyte converted from a second reaction in the lower chamber 1216 of the SATC 1210 is loadable on the test strip 1217, which can be inserted into a sample interface assembly (not shown) of the custom analyte meter 1220. The test strip 1217 includes electrical contacts that interface with electrical leads of the potentiostat to conduct the electrochemical analysis protocol (e.g., potentiometry) to measure the analyte on the test strip 1217.

FIG. 13 shows a diagram illustrating an example embodiment of an aptamer-based viral assay method 1300 using the example SATC 1210 shown in FIG. 12. The method includes a process 1310 to collect a sample from a patient (e.g., patient spits into the receptacle 1212 of the SATC 1210). The method 1300 includes a process 1320 to cap (i.e., close or seal) the receptacle 1212 with the mixing tool 1216 and mix the patient sample with the solution containing the aptamer-based assay device. The method 1300 includes a process 1330 to facilitate the antigen-aptamer / enzyme release assay over a period of time (e.g., 15 minutes), after which the process 1330 includes causing separation of the antigen-bound biological complex from the released enzyme. The method 1300 includes a process 1340 to transfer the released enzyme (e.g., in some implementations, by twisting the receptacle 1212 or the mixing tool 1216 to open a channel) from the first reaction chamber 1214 to the second reaction chamber 1218. The method 1300 includes a process 1350 to facilitate the conversion of a primary substance contained in the second reaction chamber 1218 to a target analyte via catalysis with the released enzyme transferred into the second reaction chamber 1218. The method 1300 includes a process 1360 to obtain the sample containing the target analyte (e.g., from the analyte sample chamber 1219 of the SATC 1210, which can be via a test strip that is readable by the custom analyte meter 1220).

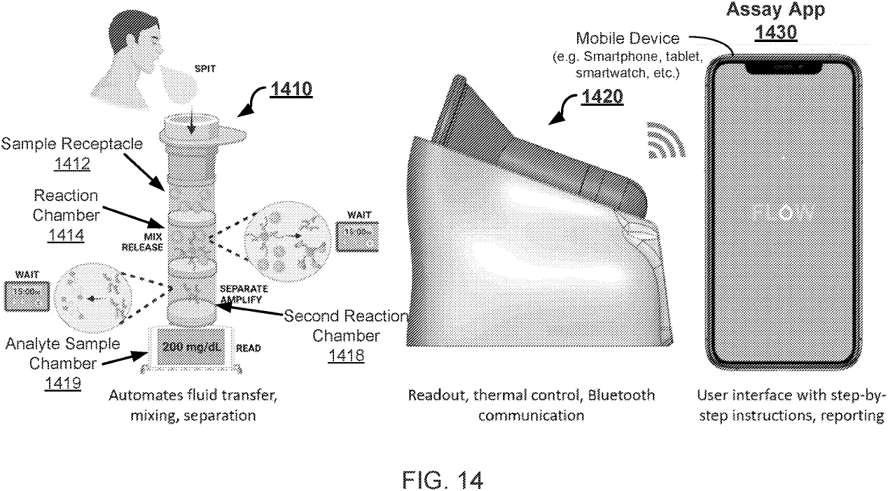

FIG. 14 shows a diagram illustrating another example embodiment of a semi-automated test container (SATC) 1410 couplable to an example embodiment of a custom analyte meter 1420, in accordance with the present technology.

The SATC 1410 includes a sample receptacle 1412 having a container with an opening to allow a patient to provide a biological sample (e.g., saliva sample, by the patient spitting in the sample receptacle 1412 to a predetermined level). The SATC 1410 includes a reaction chamber 1414, which is disposed below the sample receptacle 1412, and which can be configured to include the aptamer-based assay device, such as the example embodiment including a substrate (e.g., magnetic or polystyrene particles) conjugated with one or more aptamers (e.g., the anti-S and/or anti-N protein aptamers) initially bound to an enzyme-tagged oligonucleotide having a complementary strand with an affinity to the one or more aptamers). For example, the reaction chamber 1414 can include a solution (e.g., one or more buffers) that contains the aptamer-based assay device. In some embodiments, for example, the SATC 1410 can include a mixing tool (not shown) that includes a handle from which a shaft projects and is able to cause the churning or mixing of the patent sample with the solution containing the aptamer-based assay device in the reaction chamber 1414. The SATC 1410 includes a second reaction chamber 1418, which is disposed below the reaction chamber 1414, to facilitate conversion of a substance that is to convert to the analyte for measurement based on the enzyme released during the reaction in the reaction chamber 1414. In implementations, for example, the patient sample containing the viral antigen reacts with the aptamer-based viral assay device to bind to the one or more aptamers of the device while releasing the enzyme-tagged complementary strands into the solution. The substrate (e.g., polystyrene or magnetic particles) are separated from the released enzyme-tagged oligonucleotides; and the released enzyme-tagged oligonucleotides are separated into the second reaction chamber 1418. In some embodiments, the second reaction chamber 1418 can contain a second solution (e.g., one or more buffers) containing a primary substance that is to be converted into the analyte measurable by the custom analyte reader 1420. The converted analyte can be passed into an analyte sample chamber 1419 of the SATC 1410. In the example embodiment of the SATC 1410 shown in FIG. 14, the analyte sample chamber 1419 is able to interface with a receiving portion of the custom analyte meter 1420.

In some embodiments, for example, the custom analyte meter 1420 can include a projection member from which electrodes coupled to an electronic device (housed within the custom analyte meter 1420) can protrude through an end of the analyte sample chamber 1419 when the SATC 1410 is coupled to the custom analyte meter 1420, e.g., in order to operate an electrochemical analysis to detect the converted analyte (e.g., glucose), which corresponds to the target virus to be tested from the patient sample.

The custom analyte meter 1420 includes a heating unit to provide thermal control for the electrochemical analysis. The custom analyte meter 1420 includes an electronic unit that includes a printed circuit board (PCB) having an electrical circuit, which in some embodiments can include a potentiostat configured to control an electrochemical sensor for electroanalysis of a solution containing a converted analyte that was converted from a primary substance into the converted analyte via the released enzyme from the example aptamer-based assay. The custom analyte meter 1420 includes a power supply, which is in electrical communication with the electronic unit and/or the heating unit. In some embodiments, the custom analyte meter 1420 can include a data processing unit in communication with the electronics unit and a wireless transmitter unit in communication with the data process unit. The wireless transmitter unit can include a transceiver (Tx/Rx) to wirelessly transmit data processed by the data processing unit to a remote mobile device, e.g., a smartphone, tablet, smartwatch, etc.). The mobile device can include an assay app 1430, which is at least partially resident on the mobile device and operable to process and/or display data to a user.

In some embodiments, for example, the data processing unit can include a processor to process data, and memory in communication with the processor to store and/or buffer data. For example, the processor can include a central processing unit (CPU) or a microcontroller unit (MCU). For example, the memory can include and store processor-executable code, which when executed by the processor, configures the data processing unit to perform various operations, e.g., such as receiving information, commands, and/or data, processing information and data, and transmitting or providing information/data to another device. To support various functions of the data processing unit, the memory can store information and data, such as instructions, software, values, images, and other data processed or referenced by the processor. For example, various types of Random Access Memory (RAM) devices, Read Only Memory (ROM) devices, Flash Memory devices, and other suitable storage media can be used to implement storage functions of the memory. In some implementations, the data processing unit includes an input/output (I/O) unit to interface the processor and/or memory to other modules, units or devices, e.g., associated with the mobile device, a remote data processing system (e.g. in a network of computers, such as in the cloud), and/or other external devices. In some embodiments, the processor, memory, and/or I/O unit is in communication with the wireless communications unit, e.g., such as a transmitter (Tx) or a transmitter/receiver (Tx/Rx) unit. For example, in such embodiments, the I/O unit can interface the processor and memory with the wireless communications unit, e.g., to utilize various types of wireless interfaces compatible with typical data communication standards, which can be used in communications of the data processing unit with other devices, e.g., such as between the one or more computers in the cloud and the mobile device. The data communication standards include, but are not limited to, Bluetooth, Bluetooth low energy (BLE), Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE cellular communication methods, and parallel interfaces. In some implementations, the data processing unit can interface with other devices using a wired connection via the I/O unit. The data processing unit can also interface with other external interfaces, sources of data storage, and/or visual or audio display devices, etc. to retrieve and transfer data and information that can be processed by the processor, stored in the memory, or exhibited on an output unit of the mobile device (e.g., smartphone) or an external device.

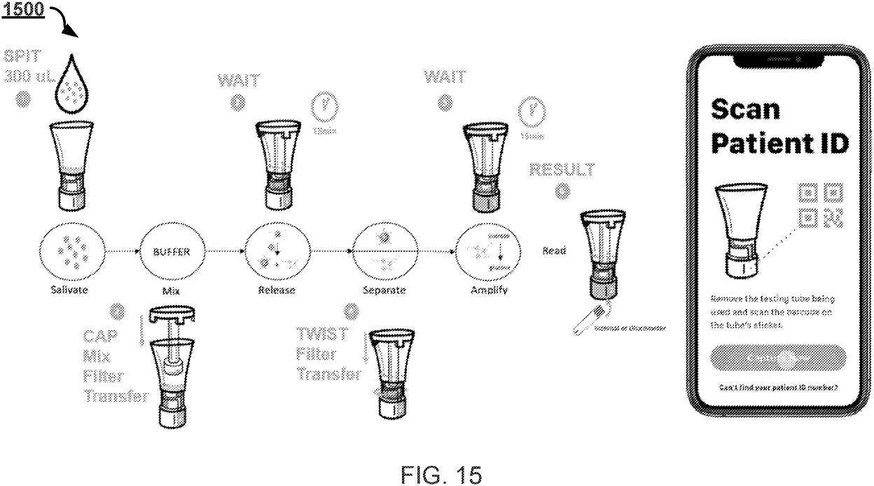

FIG. 15 shows a diagram illustrating an example embodiment of an aptamer-based viral assay method 1500, similar to the method 1300 in FIG. 13, using an example embodiment of the SATC 1210 shown in FIG. 12 and with an analyte meter including a custom analyte meter like the example custom analyte meter 1220 or 1420, or a standard glucometer. In this example of the method 1500, the entire assay process using the example SATC 1210 is illustrated and described via the app 1430 on the patient’s mobile device.

Example implementations of the aptamer-based viral assay using example embodiments of the SATC and custom analyte meter were implemented. For example, to show the reliability of the example potentiostat-based analyte (glucose) reader, example implementations of the example SARS CoV-2 assay (like that shown for the method 100′, 1300, 1500, etc.) were performed. Notably, it was recognized that stable temperatures during implementation of the aptamer-based viral assay reduces variability and improves extensibility of the platform.

### Example Optimization of Antisense Oligonucleotide Sequences for SARS-CoV-2 N Protein and S Protein

In some example implementations, the disclosed aptamer-based viral assay device using the engineered anti-N protein aptamer and anti-S protein aptamer was further investigated. For example, in some embodiments, the antisense complementary nucleotide strand configured to initially bind to the engineered anti-N protein aptamer includes a nucleic acid sequence that is at least 80% identical to nucleic acid sequence set forth in SEQ ID NO:1, SEQ ID NO:2, SEQ ID NO:3, or SEQ ID NO:4, which are listed in Table 4. For example, in some embodiments, the antisense complementary nucleotide strand configured to initially bind to the engineered anti-S protein aptamer includes a nucleic acid sequence that is at least 80% identical to nucleic acid sequence set forth in SEQ ID NO:5, SEQ ID NO:6, SEQ ID NO:7, or SEQ ID NO:8, which are listed in Table 5.

Table 4 shows example nucleic acid sequences of exemplary complementary nucleotide strand configured to initially bind to the engineered anti-N protein aptamer.

TABLE 4

| SEQ ID No. | Sequence |
|---|---|
| 1 | GACCGCCCCAGCCT |
| 2 | GACCGCCCCAG |
| 3 | GACCGCCCCAGCCTCAC |
| 4 | GACCGCCCCAGCCTCACCAA |

Table 5 shows example nucleic acid sequences of exemplary complementary nucleotide strand configured to initially bind to the engineered anti-S protein aptamer.

TABLE 5

| SEQ ID No. | Sequence |
|---|---|
| 5 | TGTCCATTAACGCCC |
| 6 | TGTCCATTAACG |
| 7 | TGTCCATTAACGCCCTTG |
| 8 | TGTCCATTAACGCCCTTGGAC |

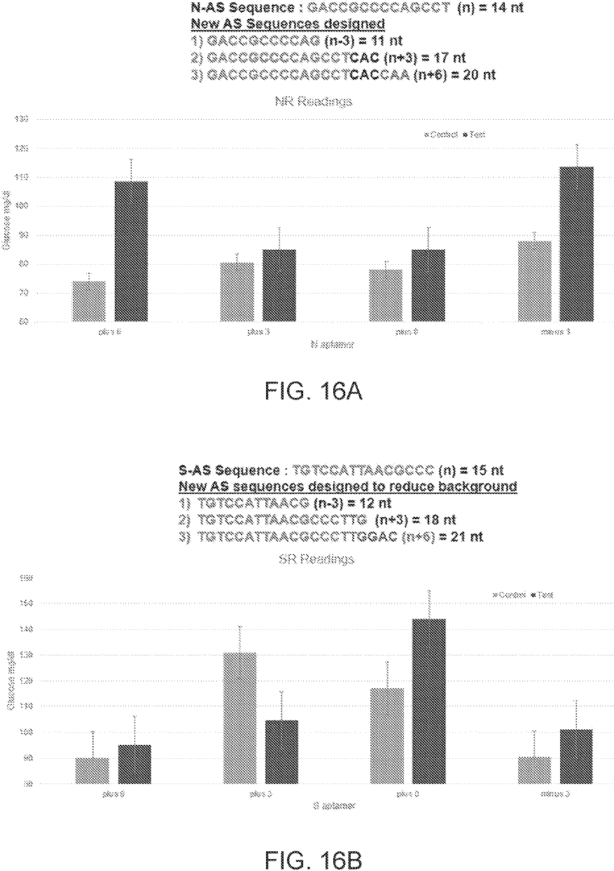

FIGS. 16A and 16B include data plots listing example antisense (oligonucleotide) sequences configured to bind to the anti-N protein aptamer and to the anti-S protein aptamer, respectively.

As shown in FIG. 16A, the three additional antisense (oligonucleotide) sequences were modified based on the engineered 14-nitrogen base sequence GACCGCCCCAGCCT, where G is guanine, C is cytosine, A is adenine, and T is thymine, which were tested with respect to the engineered anti-N protein aptamer. The three additional antisense sequences included a sequence including (i) a shorter (11-nucleotide) sequence GACCGCCCCAG, (ii) a longer (17-nucleotide) sequence GACCGCCCCAGCCTCAC, and (iii) an even longer (20-nucleotide) sequence GACCGCCCCAGCCTCACCAA. While all four engineered antisense sequences were effective, it was found that the longest (20-nucleotide) sequence performed the best for certain implementations.

As shown in FIG. 16B, the three additional antisense (oligonucleotide) sequences were modified based on the engineered 15-nitrogen base sequence TGTCCATTAACGCCC, which were tested with respect to the engineered anti-S protein aptamer. The three additional antisense sequences included a sequence including (i) a shorter (12-nucleotide) sequence TGTCCATTAACG, (ii) a longer (18-nucleotide) sequence TGTCCATTAACGCCCTTG, and (iii) an even longer (21-nucleotide) sequence TGTCCATTAACGCCCTTGGAC. While all four engineered antisense sequences were effective, it was found that the original (15-nucleotide) sequence performed the best for certain implementations.

### Example Controls in Assays

In some example implementations, the disclosed aptamer-based viral assay device can include colorimetric indicators as controls, allowing the assay operator to visually monitor the start and end of the assay.

In some implementations, an indicator for the addition of saliva to the test tube can be included in the assay. Since saliva is a colorless liquid, for example, it is beneficial to introduce a colorimetric indicator step that could serve as a visual indicator and ensure the proper addition of the specimen to the test tube. For this, Ethylidene-pNP-G7 can be used, which is a colorless substrate that produces a yellow-colored product, p-Nitrophenol (p-NP), by the enzyme alpha-amylase present in saliva. To test, two controls (buffer only) and two saliva samples were used for the assay. Yellow color from the chromophore, p-NP, is visualized only in samples containing saliva. The example results of these example colorimetric indicator implementations indicated that the chemicals did not interfere with the downstream assay conditions. It is noted that this aspect incorporated in the disclosed assay can minimize the possibility of false-negative results that could potentially arise due to mistakes made during the addition of saliva to the test.

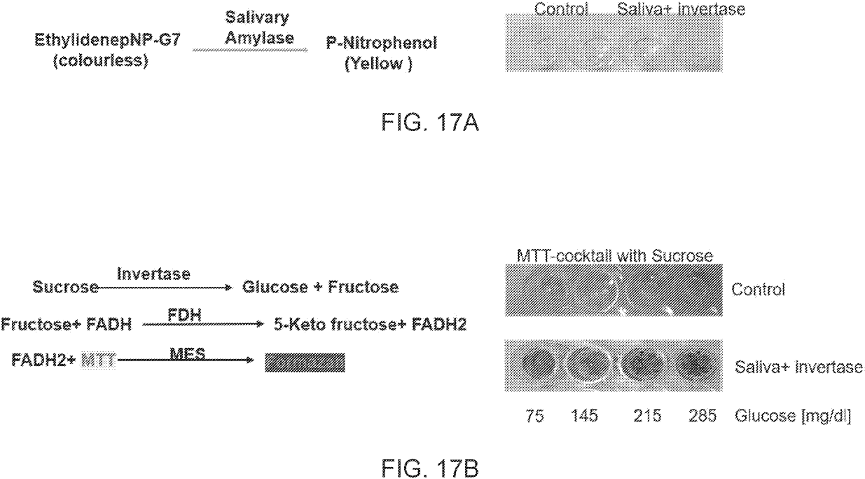

FIG. 17A shows an image depicting an example result of a colorimetric control process for indicating the successful addition of saliva to the assay receptacle. The image shows how the Ethylidene-pNP-G7 was able to produce the yellow-colored product, p-Nitrophenol.

In some implementations, an indicator for the conversion of the sucrose to glucose and fructose in the presence of the Invertase enzyme can be included in the assay. For example, an additional colorimetric indicator was added that can signal the completion of the assay to the user and prompt them to get the results by using the glucometer. For this, a modified version of the MTT assay was used. The principle is as follows: Sucrose is first converted to Glucose and Fructose in the presence of the Invertase enzyme. The product- Fructose is next converted to 5-Ketofructose by the enzyme, Formate dehydrogenase (FDH)- and produces the reduced form of Flavin Adenine Dinucleotide (FADH₂). The MTT reagent then reacts with FADH₂ in the presence of MES (Phenazine methosulfate) to form a purple-colored product, Formazan, that can be detected visually. The upper control panel shows the yellow color of the MTT reagent and the Sucrose solution in absence of invertase. The purple color (Formazan) is only formed when Invertase is added to the reaction. The increase in the intensity of the purple color corresponds to the increasing concentration of the added Invertase (1, 2.5, 5, and 10 nM) in the reaction wells, respectively. This also corresponds to the proportional increase in glucose production as indicated by the glucometer reading, and additionally proved that the colorimetric assay did not interfere with the glucometer measurement.

FIG. 17B shows an image depicting an example result of a colorimetric control process for indicating the conversion of the sucrose to glucose and fructose in the presence of the invertase enzyme.

### Low-temperature Invertase

In some embodiments, the enzyme of the aptamer-based viral assay device can include a low temperature enzyme capable of catalyzing the substance at a temperature in a range of 20° C. to 35° C. In some example implementations, optimization of a low-temperature invertase enzyme was performed to utilize the assay under lower temperature conditions. A comparison between the low-temperature bacterial Invertase enzyme and the high-temperature yeast Invertase enzyme was studied. The optimal temperature for the enzymatic activity of the yeast enzyme is ~60° C. and can be an impediment to use in low resource areas. This can be overcome by using the bacterial Invertase enzyme that has optimal enzymatic activity at room temperature. However, as noted in the table, the signal-to-noise (S/N) ratio for the bacterial enzyme in this example was lower as compared to the yeast enzyme.

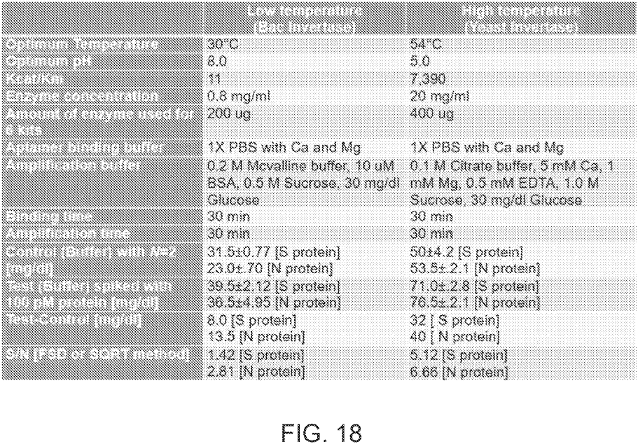

FIG. 18 shows a table depicting a comparison between a low-temperature bacterial invertase enzyme and a high-temperature yeast invertase enzyme.

### Lyophilization

In some embodiments, the disclosed aptamer-based viral assay device and method can include lyophilization. Lyophilization can improve the shelf-life of the assay. In some embodiments, the assay reagents are lyophilized and stored in powder form in both reaction vessels. In some example implementations, N or S magnetic bead lyophilized kits (e.g., magnetic beads conjugated with antisense/invertase-aptamer complex) were prepared in an optimized lyophilization buffer for N and S respectively. The standard buffer for N kits containing 4% trehalose, 5% DMSO, 0.5% Tween and for Skits containing 4% Trehalose, 5% DMSO. During the optimization process, a range of trehalose, Tween, and DMSO concentrations were evaluated. In addition, the contribution of BSA and other polymers to the stability of kits were assessed. Assay mixtures were aliquoted 60 µL/tube (200 µg of beads), frozen in liquid nitrogen, and lyophilized at 0.01 mbar for at least 16 h in Labconco lyophilizer. For detection, a dried tube was resuspended in 60 µL of DPBS (1x PBS pH 7.4 with 0.90 mM Calcium and 0.50 mM Magnesium), and the antigen was added as described. Assay performance was compared to one day old, stored sample at 4° C. The amplification assay buffer included 0.1 M Citrate buffer pH 5.0, 10 µM BSA, 13 mM Glucose, 1 M sucrose, 0.5 mM Calcium and 1 mM Magnesium and 0.5 mM EDTA.

### EXAMPLES

In some embodiments in accordance with the present technology (Example A1), a point-of-care (POC) aptamer-based COVID-19 assay device includes a magnetic bead; a biochemical complex comprising a N or S protein specific aptamer conjugated to the magnetic bead, wherein the N or S protein specific aptamer is pre-hybridized with a complementary DNA strand attached to invertase; wherein, upon mixing the device with SARS-CoV-2 in a container and binding of the device with SARS-CoV-2, the invertase is released from the biochemical complex, such that a magnet interacting with the container removes the magnetic bead bound to the SARS-CoV-2, and the unbound invertase react is extractable and able to react in a subsequent reaction with sucrose to generate glucose that is directly readout using a glucometer, wherein the glucose concentration is correlatable with SARS-CoV-2 copy number.

In some embodiments in accordance with the present technology (Example A2), a point-of-care (POC) aptamer-based COVID-19 assay method includes forming an analytical bio-complex device by conjugating a N or S protein specific aptamer to a magnetic bead and pre-hybridizing the N or S protein specific aptamer with a complementary DNA strand attached to invertase; mixing the analytical bio-complex device with a biological sample collected from a patient to test for SARS-CoV-2, wherein the mixing allows for binding of SARS-CoV-2 to the analytical bio-complex device, and wherein upon binding of the analytical bio-complex device with SARS-CoV-2, the invertase is released; applying a magnetic field to the mixture, wherein the magnetic field causes removal of the magnetic bead bound to the SARS-CoV-2 from the released invertase; obtaining the released invertase; reacting the obtained invertase with sucrose to generate glucose that can be directly read using a glucometer; and correlating a glucose concentration with SARS-CoV-2 copy number.

In some embodiments in accordance with the present technology (Example B1), an aptamer-based viral assay device includes a substrate including a surface; and a biochemical complex conjugated to the surface of the substrate, the biochemical complex comprising an aptamer that is initially bound to a complementary strand of nucleotides that attaches an enzyme, the aptamer corresponding to an antigen of a virus that has a higher binding affinity to the aptamer than the complementary strand of nucleotides, wherein, when the device is exposed to a solution containing the virus, the biochemical complex is configured to release the complementary strand of nucleotides that attaches the enzyme from the aptamer to the solution and bind the antigen of the virus to the aptamer to form a modified biochemical complex conjugated to the surface of the substrate, wherein the enzyme that is released to the solution is capable of converting a substance to an analyte that is measurable by a remote analyte meter to correlate with a parameter of the virus in the solution.

Example B2 includes the device of any of examples B1-B15, wherein the complementary strand of nucleotides, which is initially bound the aptamer, attaches a plurality of the enzyme.

Example B3 includes the device of example B2 or any of examples B 1-B 15, wherein the complementary strand of nucleotides includes a first nucleic acid strand and a second nucleic acid strand, wherein the first nucleic acid strand includes a first nucleotide sequence having a first region that binds to the aptamer and having a second region that binds to the second nucleic acid strand, wherein the first nucleic acid strand binds at least one of the plurality of the enzyme proximate the second region, and wherein the second nucleic acid strand includes a second nucleotide sequence having a first portion that binds to the second region of the first nucleic acid strand, wherein the second nucleic acid strand binds at least another one of the plurality of the enzyme.

Example B4 includes the device of example B2 or any of examples B 1-B 15, wherein the complementary strand of nucleotides includes a first nucleic acid strand, a second nucleic acid strand, and a third nucleic acid strand, wherein the first nucleic acid strand includes a first nucleotide sequence having a first region that binds to the aptamer and having a second region that binds to the second nucleic acid strand, wherein the second nucleic acid strand includes a second nucleotide sequence having a first portion that binds to the second region of the first nucleic acid strand and having a second portion that binds to the third nucleic acid strand, wherein the second nucleic acid strand binds at least one of the plurality of the enzyme proximate the second portion, wherein the third nucleic acid strand includes a third nucleotide sequence having a first section that binds to the second portion of the second nucleic acid strand, and wherein the third nucleic acid strand binds at least another one of the plurality of the enzyme.

Example B5 includes the device of any of examples B 1-B 15, wherein the virus is SARS CoV-2 virus, wherein the antigen of the SARS CoV-2 virus includes one or both of a nucleocapsid protein (N protein) and a spike surface glycoprotein (S protein) of the SARS CoV-2 virus, and wherein aptamer includes one or more aptamers comprising one or both of an anti-S protein aptamer and an anti-N protein aptamer that correspond to the N protein and the S protein, respectively.

Example B6 includes the device of example B5 or any of examples B 1-B 15, wherein the complementary strand of nucleotides includes one or both of a first complementary strand of nucleotides configured to initially bind to the anti-S protein aptamer and a second complementary strand of nucleotides configured to initially bind to the anti-N protein aptamer.

Example B7a includes the device of example B6 or any of examples B 1-B 15, wherein the first complementary strand of nucleotides comprises a nucleic acid sequence that is at least 80% identical to a nucleic acid sequence including one of TGTCCATTAACGCCC, TGTCCATTAACG, TGTCCATTAACGCCCTTG, or TGTCCATTAACGCCCTTGGAC.

Example B7b includes the device of example B6 or any of examples B 1-B 15, wherein the first complementary strand of nucleotides comprises a nucleic acid sequence that is at least 90% identical to a nucleic acid sequence including one of TGTCCATTAACGCCC, TGTCCATTAACG, TGTCCATTAACGCCCTTG, or TGTCCATTAACGCCCTTGGAC.

Example B7c includes the device of example B6 or any of examples B 1-B 15, wherein the first complementary strand of nucleotides comprises a nucleic acid sequence that includes one of TGTCCATTAACGCCC, TGTCCATTAACG, TGTCCATTAACGCCCTTG, or TGTCCATTAACGCCCTTGGAC.

Example B8a includes the device of example B6 or any of examples B1-B15, wherein the second complementary strand of nucleotides comprises a nucleic acid sequence that is at least 80% identical to a nucleic acid sequence including one of GACCGCCCCAGCCT, GACCGCCCCAG, GACCGCCCCAGCCTCAC, or GACCGCCCCAGCCTCACCAA.

Example B8b includes the device of example B6 or any of examples B 1-B 15, wherein the second complementary strand of nucleotides comprises a nucleic acid sequence that is at least 90% identical to a nucleic acid sequence including one of GACCGCCCCAGCCT, GACCGCCCCAG, GACCGCCCCAGCCTCAC, or GACCGCCCCAGCCTCACCAA.

Example B8c includes the device of example B6 or any of examples B1-B15, wherein the second complementary strand of nucleotides comprises a nucleic acid sequence that includes one of GACCGCCCCAGCCT, GACCGCCCCAG, GACCGCCCCAGCCTCAC, or GACCGCCCCAGCCTCACCAA.

Example B9 includes the device of any of examples B 1-B15, wherein the enzyme includes invertase, wherein the analyte includes glucose, wherein the substance includes sucrose, and wherein the remote analyte meter includes a glucometer, such that the invertase is capable of facilitating conversion of the sucrose to glucose that is measurable by the glucometer, wherein a glucose concentration measurable by the glucometer correlates with a copy number of the SARS CoV-2 virus.

Example B10 includes the device of any of examples B1-B15, wherein the substrate includes a magnetic particle.

Example B11 includes the device of example B10 or any of examples B 1-B 15, wherein the device is operable to allow collection of the released complementary strand attached to the enzyme from the solution through magnetic separation of the modified biochemical complex conjugated to the surface of the magnetic particle and the enzyme.

Example B 12 includes the device of any of examples B 1-B 15, wherein the substrate includes a polystyrene particle configured to have a size in a range of at least 1 µm.

Example B13 includes the device of any of examples B1-B15, wherein the biochemical complex includes a includes biotin at a terminus of the aptamer that is bound to the surface of the substrate, and wherein the surface of the substrate includes a layer comprising streptavidin.

Example B14 includes the device of any of examples B 1-B15, wherein the surface of the substrate is modified with one or more blocking proteins to prevent binding of other substances to the substrate.

Example B15 includes the device of any of examples B 1-B14, wherein the enzyme includes a low temperature enzyme capable of catalyzing the substance at a temperature in a range of 20° C. to 35° C.

In some embodiments in accordance with the present technology (Example B16), a viral assay kit device includes a sample receptacle comprising a container having one or more sidewalls and a bottom wall with an opening to receive a biological sample in the container; a reaction chamber disposed adjacent to the sample receptacle and configured to include an aptamer-based viral assay device in a solution contained in the reaction chamber, wherein the aptamer-based viral assay includes a substrate and a biochemical complex conjugated to a surface of the substrate, the biochemical complex comprising an aptamer that is initially bound to a complementary strand of nucleotides that attaches an enzyme, the aptamer corresponding to a target antigen of a virus that has a higher binding affinity to the aptamer than the complementary strand of nucleotides, wherein, when the aptamer-based viral assay device is operable to release the complementary strand of nucleotides that attaches the enzyme in the solution based on a binding of the target antigen of the virus in the biological sample, wherein the biochemical complex is configured to release the complementary strand of nucleotides that attaches the enzyme from the aptamer to the solution and bind the target antigen of the virus to the aptamer to form a modified biochemical complex conjugated to the surface of the substrate; a mixing tool operable to mix the biological sample from the container with the solution containing the aptamer-based viral assay device in the reaction chamber; and a second reaction chamber disposed adjacent to the reaction chamber and configured to include a primary substance in a fluid, wherein, when the enzyme is exposed to the primary substance, the enzyme facilitates conversion of the primary substance in the second reaction chamber to an analyte.

Example B17 includes the device of any of examples B 16-B28, wherein the analyte is measurable by a remote analyte meter device to correlate with a parameter of the virus in the biological sample.

Example B18 includes the device of example B17 or any of examples B16-B28, further comprising an analyte meter device operable to measure the analyte, wherein the analyte meter device includes an electronics unit comprising a potentiostat configured to operate an electrochemical analysis on the analyte when the analyte is brought in contact with electrodes in electrical communication with the potentiostat.

Example B19 includes the device of example B18 or any of examples B16-B28, wherein the analyte meter device includes a data processing unit in communication with the electronics unit, and a wireless communications unit in communication with the data processing unit, wherein the data processing unit is operable to process signal data from the potentiostat and evaluate a parameter of the analyte that correlates with the parameter of the virus in the biological sample.

Example B20 includes the device of example B19 or any of examples B 16-B28, further comprising a software application able to reside in memory on a mobile device, the software application comprising code executable by a processor of the mobile device to cause the mobile device to receive data transmitted from the analyte meter device, process the received data to determine one or both of the parameter of the analyte and the parameter of the virus in the biological sample, and display a user interface on a display screen of the mobile device to present information pertaining to the determined one or both of the parameter of the analyte and the parameter of the virus.

Example B21 includes the device of any of examples B16-B28, wherein the mixing tool comprises a top portion from which a shaft projects, wherein the top portion is configured to cover the opening of the sample receptacle and the shaft is configured to perforate at least a portion of the bottom wall to cause the biological sample to be exposed with the reaction chamber.

Example B22 includes the device of any of examples B 16-B28, wherein the fluid in the second reaction chamber includes one or more buffers containing the primary substance that is to be converted into the analyte.

Example B23 includes the device of any of examples B16-B28, further comprising an analyte sample chamber disposed adjacent to the second reaction chamber, wherein the analyte sample chamber is configured to the analyte after the conversion.

Example B24 includes the device of any of examples B 16-B28, wherein the complementary strand of nucleotides, which is initially bound the aptamer, attaches a plurality of the enzyme.

Example B25 includes the device of example B24 or any of examples B16-B28, wherein the complementary strand of nucleotides includes a first nucleic acid strand and a second nucleic acid strand, wherein the first nucleic acid strand includes a first nucleotide sequence having a first region that binds to the aptamer and having a second region that binds to the second nucleic acid strand, wherein the first nucleic acid strand binds at least one of the plurality of the enzyme proximate the second region, and wherein the second nucleic acid strand includes a second nucleotide sequence having a first portion that binds to the second region of the first nucleic acid strand, wherein the second nucleic acid strand binds at least another one of the plurality of the enzyme.

Example B26 includes the device of example B24 or any of examples B16-B28, wherein the complementary strand of nucleotides includes a first nucleic acid strand, a second nucleic acid strand, and a third nucleic acid strand, wherein the first nucleic acid strand includes a first nucleotide sequence having a first region that binds to the aptamer and having a second region that binds to the second nucleic acid strand, wherein the second nucleic acid strand includes a second nucleotide sequence having a first portion that binds to the second region of the first nucleic acid strand and having a second portion that binds to the third nucleic acid strand, wherein the second nucleic acid strand binds at least one of the plurality of the enzyme proximate the second portion, wherein the third nucleic acid strand includes a third nucleotide sequence having a first section that binds to the second portion of the second nucleic acid strand, and wherein the third nucleic acid strand binds at least another one of the plurality of the enzyme.

Example B27 includes the device of any of examples B16-B28, wherein the virus is SARS CoV-2 virus, wherein the antigen of the SARS CoV-2 virus includes one or both of a nucleocapsid protein (N protein) and a spike surface glycoprotein (S protein) of the SARS CoV-2 virus, and wherein aptamer includes one or more aptamers comprising one or both of an anti-S protein aptamer and an anti-N protein aptamer that correspond to the N protein and the S protein, respectively, and/or wherein: the enzyme includes invertase, the analyte includes glucose, and/or the primary substance includes sucrose.

Example B28 includes the device of any of examples B16-B27, wherein the aptamer-based viral assay device includes the aptamer-based viral assay device of any of examples B1-B15.

In some embodiments in accordance with the present technology (Example B29), an aptamer-based viral assay method includes forming an assay device by conjugating a biochemical complex to a substrate, the biochemical complex comprising an aptamer that is initially bound to a complementary strand of nucleotides that attaches an enzyme, the aptamer corresponding to a target antigen of a virus that has a higher binding affinity to the aptamer than the complementary strand of nucleotides; mixing a solution comprising the assay device with a biological sample collected from a patient to test for the virus, wherein the mixing the assay device with the biological sample facilitates binding of the target antigen of the virus with the aptamer to form a modified biochemical complex conjugated to the substrate, and, upon binding of the target antigen with the aptamer, the biochemical complex releases the complementary strand of nucleotides that attaches the enzyme in a solution; separating the assay device having the modified biochemical complex conjugated to the substrate from the complementary strand of nucleotides that attaches the enzyme released in the solution; and reacting a primary substance with the enzyme to cause the primary substance to convert to an analyte measurable with an analyte meter such that a parameter of the analyte can be measured by the analyte meter and correlate with a parameter of the virus from the biological sample.

Example B30 includes the method of any of examples B29-B33, wherein the complementary strand of nucleotides, which is initially bound the aptamer, attaches a plurality of the enzyme.

Example B31 includes the method of any of examples B29-B33, wherein the enzyme includes a low temperature enzyme capable of catalyzing the primary substance at a temperature in a range of 20° C. to 35° C.

Example B32 includes the method of any of examples B29-B33, further comprising lyophilizing one or more of the aptamer, the complementary strand of nucleotides that attaches the enzyme, or the biochemical complex conjugated to the substrate prior to the forming the assay device.

Example B33 includes the method of any of examples B29-B32, wherein the assay device includes the aptamer-based viral assay of any of examples B1-B15; and/or wherein the method is implemented using the viral assay kit device of any of examples B15-B28.

In some embodiments in accordance with the present technology (Example B34), a point-of-care (POC) aptamer-based COVID-19 assay device includes a magnetic bead; and a biochemical complex comprising one or both of a nucleocapsid protein (N protein) specific aptamer and a spike surface glycoprotein (S protein) specific aptamer conjugated to the magnetic bead, wherein the one or both of the N protein specific aptamer and the S protein specific aptamer is pre-hybridized with a complementary DNA strand attached to invertase; wherein, upon mixing the device with a sample containing SARS-CoV-2 virus in a container, the complementary DNA strand attached to the invertase is released from the biochemical complex and the one or both of the N protein specific aptamer and the S protein specific aptamer is bound to one or both of N protein antigen and S protein antigen of the SARS-CoV-2 virus, respectively, to form a modified biochemical complex conjugated to the magnetic bead, wherein, upon applying a magnet field at the container, the magnetic bead conjugated with the modified biochemical complex separates from the released complementary DNA strand attached to the invertase, and wherein the separated invertase is able to react with sucrose to generate glucose that is able to be directly measured using a glucometer, wherein the measured glucose is correlatable with a parameter of the SARS-CoV-2 virus from the sample.

In some embodiments in accordance with the present technology (Example B35), a point-of-care (POC) aptamer-based COVID-19 assay method includes forming an assay device by conjugating one or both of a nucleocapsid protein (N protein) specific aptamer and a spike surface glycoprotein (S protein) specific aptamer to a magnetic bead and pre-hybridizing the one or both of the N protein specific aptamer and the S protein specific aptamer with a complementary DNA strand attached to invertase; mixing the assay device with a biological sample collected from a patient to test for SARS-CoV-2 virus, wherein the mixing the assay device with the biological sample facilitates binding of the SARS-CoV-2 virus to the assay device by the one or both of the N protein specific aptamer and the S protein specific aptamer binding to one or both of N protein antigen and S protein antigen of the SARS-CoV-2 virus, respectively, and wherein, upon binding of the assay device with the SARS-CoV-2 virus, the complementary DNA strand attached to the invertase is released; applying a magnetic field to the mixture to separate the magnetic bead conjugated with the modified biochemical complex from the complementary DNA strand attached to the invertase; reacting the invertase with sucrose to generate glucose that can be directly read using a glucometer; and correlating a glucose concentration with a parameter of the SARS-CoV-2 virus from the biological sample.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. An aptamer-based viral assay device, comprising:
a substrate including a surface; and
a biochemical complex conjugated to the surface of the substrate, the biochemical complex comprising an aptamer that is initially bound to a complementary strand of nucleotides that attaches an enzyme, the aptamer corresponding to an antigen of a virus that has a higher binding affinity to the aptamer than the complementary strand of nucleotides,
wherein, when the device is exposed to a solution containing the virus, the biochemical complex is configured to release the complementary strand of nucleotides that attaches the enzyme from the aptamer to the solution and bind the antigen of the virus to the aptamer to form a modified biochemical complex conjugated to the surface of the substrate,
wherein the enzyme that is released to the solution is capable of converting a substance to an analyte that is measurable by a remote analyte meter to correlate with a parameter of the virus in the solution.

2. The device of claim 1, wherein the complementary strand of nucleotides, which is initially bound the aptamer, attaches a plurality of the enzyme.

3. The device of claim 2, wherein the complementary strand of nucleotides includes a first nucleic acid strand and a second nucleic acid strand, wherein the first nucleic acid strand includes a first nucleotide sequence having a first region that binds to the aptamer and having a second region that binds to the second nucleic acid strand, wherein the first nucleic acid strand binds at least one of the plurality of the enzyme proximate the second region, and wherein the second nucleic acid strand includes a second nucleotide sequence having a first portion that binds to the second region of the first nucleic acid strand, wherein the second nucleic acid strand binds at least another one of the plurality of the enzyme.

4. The device of claim 2, wherein the complementary strand of nucleotides includes a first nucleic acid strand, a second nucleic acid strand, and a third nucleic acid strand, wherein the first nucleic acid strand includes a first nucleotide sequence having a first region that binds to the aptamer and having a second region that binds to the second nucleic acid strand, wherein the second nucleic acid strand includes a second nucleotide sequence having a first portion that binds to the second region of the first nucleic acid strand and having a second portion that binds to the third nucleic acid strand, wherein the second nucleic acid strand binds at least one of the plurality of the enzyme proximate the second portion, wherein the third nucleic acid strand includes a third nucleotide sequence having a first section that binds to the second portion of the second nucleic acid strand, and wherein the third nucleic acid strand binds at least another one of the plurality of the enzyme.

5. The device of claim 4, wherein the virus is SARS CoV-2 virus, wherein the antigen of the SARS CoV-2 virus includes one or both of a nucleocapsid protein (N protein) and a spike surface glycoprotein (S protein) of the SARS CoV-2 virus, and wherein aptamer includes one or more aptamers comprising one or both of an anti-S protein aptamer and an anti-N protein aptamer that correspond to the N protein and the S protein, respectively.

6. The device of claim 5, wherein the complementary strand of nucleotides includes one or both of a first complementary strand of nucleotides configured to initially bind to the anti-S protein aptamer and a second complementary strand of nucleotides configured to initially bind to the anti-N protein aptamer.

7. The device of claim 6, wherein the first complementary strand of nucleotides comprises a sequence including one of TGTCCATTAACGCCC (SEQ ID NO: 5), TGTCCATTAACG (SEQ ID NO: 6), TGTCCATTAACGCCCTTG (SEQ ID NO: 7), or TGTCCATTAACGCCCTTGGAC (SEQ ID NO: 8).

8. The device of claim 6, wherein the second complementary strand of nucleotides comprises a sequence including one of GACCGCCCCAGCCT (SEQ ID NO: 1), GACCGCCCCAG (SEQ ID NO: 2), GACCGCCCCAGCCTCAC (SEQ ID NO: 3), or GACCGCCCCAGCCTCACCAA (SEQ ID NO: 4).

9. (canceled)

10. The device of claim 1, wherein the substrate includes a magnetic particle.

11. The device of claim 10, wherein the device is operable to allow collection of the released complementary strand attached to the enzyme from the solution through magnetic separation of the modified biochemical complex conjugated to the surface of the magnetic particle and the enzyme.

12. (canceled)

13. (canceled)

14. (canceled)

15. (canceled)

16. A viral assay kit device, comprising:
a sample receptacle comprising a container having one or more sidewalls and a bottom wall with an opening to receive a biological sample in the container;
a reaction chamber disposed adjacent to the sample receptacle and configured to include an aptamer-based viral assay device in a solution contained in the reaction chamber, wherein the aptamer-based viral assay includes a substrate and a biochemical complex conjugated to a surface of the substrate, the biochemical complex comprising an aptamer that is initially bound to a complementary strand of nucleotides that attaches an enzyme, the aptamer corresponding to a target antigen of a virus that has a higher binding affinity to the aptamer than the complementary strand of nucleotides, wherein, when the aptamer-based viral assay device is operable to release the complementary strand of nucleotides that attaches the enzyme in the solution based on a binding of the target antigen of the virus in the biological sample, wherein the biochemical complex is configured to release the complementary strand of nucleotides that attaches the enzyme from the aptamer to the solution and bind the target antigen of the virus to the aptamer to form a modified biochemical complex conjugated to the surface of the substrate;
a mixing tool operable to mix the biological sample from the container with the solution containing the aptamer-based viral assay device in the reaction chamber; and
a second reaction chamber disposed adjacent to the reaction chamber and configured to include a primary substance in a fluid, wherein, when the enzyme is exposed to the primary substance, the enzyme facilitates conversion of the primary substance in the second reaction chamber to an analyte.

17. The device of claim 16, wherein the analyte is measurable by a remote analyte meter device to correlate with a parameter of the virus in the biological sample.

18. The device of claim 17, further comprising:
an analyte meter device operable to measure the analyte, wherein the analyte meter device includes an electronics unit comprising a potentiostat configured to operate an electrochemical analysis on the analyte when the analyte is brought in contact with electrodes in electrical communication with the potentiostat, wherein the analyte meter device includes a data processing unit in communication with the electronics unit, and a wireless communications unit in communication with the data processing unit, wherein the data processing unit is operable to process signal data from the potentiostat and evaluate a parameter of the analyte that correlates with the parameter of the virus in the biological sample.

19. (canceled)

20. (canceled)

21. The device of claim 16, wherein the mixing tool comprises a top portion from which a shaft projects, wherein the top portion is configured to cover the opening of the sample receptacle and the shaft is configured to perforate at least a portion of the bottom wall to cause the biological sample to be exposed with the reaction chamber.

22. (canceled)

23. The device of claim 16, further comprising:
an analyte sample chamber disposed adjacent to the second reaction chamber, wherein the analyte sample chamber is configured to the analyte after the conversion.

24. The device of claim 16, wherein the complementary strand of nucleotides, which is initially bound the aptamer, attaches a plurality of the enzyme.

25. (canceled)

26. The device of claim 24, wherein the complementary strand of nucleotides includes a first nucleic acid strand, a second nucleic acid strand, and a third nucleic acid strand, wherein the first nucleic acid strand includes a first nucleotide sequence having a first region that binds to the aptamer and having a second region that binds to the second nucleic acid strand, wherein the second nucleic acid strand includes a second nucleotide sequence having a first portion that binds to the second region of the first nucleic acid strand and having a second portion that binds to the third nucleic acid strand, wherein the second nucleic acid strand binds at least one of the plurality of the enzyme proximate the second portion, wherein the third nucleic acid strand includes a third nucleotide sequence having a first section that binds to the second portion of the second nucleic acid strand, and wherein the third nucleic acid strand binds at least another one of the plurality of the enzyme.

27. The device of claim 26, wherein the virus is SARS CoV-2 virus, wherein the antigen of the SARS CoV-2 virus includes one or both of a nucleocapsid protein (N protein) and a spike surface glycoprotein (S protein) of the SARS CoV-2 virus, and wherein aptamer includes one or more aptamers comprising one or both of an anti-S protein aptamer and an anti-N protein aptamer that correspond to the N protein and the S protein, respectively.

28. (canceled)

29. An aptamer-based viral assay method, comprising:
forming an assay device by conjugating a biochemical complex to a substrate, the biochemical complex comprising an aptamer that is initially bound to a complementary strand of nucleotides that attaches an enzyme, the aptamer corresponding to a target antigen of a virus that has a higher binding affinity to the aptamer than the complementary strand of nucleotides;
mixing a solution comprising the assay device with a biological sample collected from a patient to test for the virus, wherein the mixing the assay device with the biological sample facilitates binding of the target antigen of the virus with the aptamer to form a modified biochemical complex conjugated to the substrate, and, upon binding of the target antigen with the aptamer, the biochemical complex releases the complementary strand of nucleotides that attaches the enzyme in a solution;
separating the assay device having the modified biochemical complex conjugated to the substrate from the complementary strand of nucleotides that attaches the enzyme released in the solution; and
reacting a primary substance with the enzyme to cause the primary substance to convert to an analyte measurable with an analyte meter such that a parameter of the analyte can be measured by the analyte meter and correlate with a parameter of the virus from the biological sample.

30. The method of claim 29, wherein the complementary strand of nucleotides, which is initially bound the aptamer, attaches a plurality of the enzyme.

31. The method of claim 29, wherein the enzyme includes a low temperature enzyme capable of catalyzing the primary substance at a temperature in a range of 20° C. to 35° C.

32. The method of claim 29, further comprising:
lyophilizing one or more of the aptamer, the complementary strand of nucleotides that attaches the enzyme, or the biochemical complex conjugated to the substrate prior to the forming the assay device.

33. (canceled)

34. (canceled)

35. (canceled)


SEQUENCE LISTING (14 sequences)

SEQ ID NO: 1 - DNA, 14 bp, Artificial Sequence
  Synthetic
gaccgccccagcct

SEQ ID NO: 2 - DNA, 11 bp, Artificial Sequence
  Synthetic
gaccgccccag

SEQ ID NO: 3 - DNA, 17 bp, Artificial Sequence
  Synthetic
gaccgccccagcctcac

SEQ ID NO: 4 - DNA, 20 bp, Artificial Sequence
  Synthetic
gaccgccccagcctcaccaa

SEQ ID NO: 5 - DNA, 15 bp, Artificial Sequence
  Synthetic
tgtccattaacgccc

SEQ ID NO: 6 - DNA, 12 bp, Artificial Sequence
  Synthetic
tgtccattaacg

SEQ ID NO: 7 - DNA, 18 bp, Artificial Sequence
  Synthetic
tgtccattaacgcccttg

SEQ ID NO: 8 - DNA, 21 bp, Artificial Sequence
  Synthetic
tgtccattaacgcccttggac

SEQ ID NO: 9 - DNA, 94 bp, Artificial Sequence
  Synthetic
  modified_base (1)..(1): Biotin
ttttttgcaatggtacggtacttccggatgcggaaactggctaattggtgaggctggggcggtcgtgcagcaaaagtgcacgctactttgctaa

SEQ ID NO: 10 - DNA, 26 bp, Artificial Sequence
  Synthetic
  modified_base (1)..(1): Thiol
ttttttttttttgaccgccccagcct

SEQ ID NO: 11 - DNA, 57 bp, Artificial Sequence
  Synthetic
  modified_base (1)..(1): Biotin
ttttttcagcaccgaccttgtgctttgggagtgctggtccaagggcgttaatggaca

SEQ ID NO: 12 - DNA, 27 bp, Artificial Sequence
  Synthetic
  modified_base (1)..(1): Thiol
tttttttttttttgtccattaacgccc

SEQ ID NO: 13 - DNA, 15 bp, Artificial Sequence
  Synthetic
gcaatggtacggtac

SEQ ID NO: 14 - DNA, 14 bp, Artificial Sequence
  Synthetic
cagcaccgaccttg